(19) United States
(12) Patent Application Publication
(10) Pub. No.: US 2024/0041499 A1
(43) Pub. Date: Feb. 8, 2024

(54) COMPOSITE MATERIAL DEVICES FOR CONNECTING AN ORTHOPEDIC ROD TO A PEDICLE SCREW AND SUPPLEMENTAL TOOLS

(71) Applicant: CarboFix Spine Inc., Ocean Isle Beach, NC (US)

(72) Inventors: Mordechay BEYAR, Tel Aviv (IL); Oren GLOBERMAN, Kfar Shmaryahu (IL)

(73) Assignee: CarboFix Spine Inc., Ocean Isle Beach, NC (US)

(21) Appl. No.: 18/033,346

(22) PCT Filed: Oct. 21, 2021
(86) PCT No.: PCT/IB2021/059727

§ 371 Date: Apr. 23, 2023

Related U.S. Application Data
(60) Provisional application No. 63/104,709, filed on Oct. 23, 2020.
(60) Provisional application No. 63/210,073, filed on Jun. 14, 2021.

(51) Int. Cl. A61B 17/70

(52) U.S. Cl. CPC A61B 17/7032; A61B 17/7037; A61B 2017/00964

(57) ABSTRACT

A composite material device for connecting an orthopedic rod to a pedicle screw may include: a housing having an annular body including: two opposite concave indents at a proximal end of the housing and in a longitudinal direction along the housing, the concave indents configured to receive an orthopedic rod; and an internal thread on an internal surface of a proximal portion of the housing; an adapter to lock at least a portion of a pedicle screw head within a distal portion of the housing; and an internal bolt including an external thread configured to mate with the internal thread of the housing, the internal bolt being configured to be screwed into the proximal portion of the housing so as to lock the orthopedic rod between the adapter and the internal bolt; wherein the housing, the adapter and the internal bolt comprise composite material. Supplemental tools for handling the device are disclosed.

### FIELD OF THE INVENTION

The present invention relates to the field of orthopedic implants and, more particularly, to composite material orthopedic implants.

### BACKGROUND OF THE INVENTION

Orthopedic rods and pedicle screws are well known in the art. However, devices for connecting orthopedic rods to pedicle screws are typically made of metal. Metal components of orthopedic implants may be relatively heavy. Metal components of orthopedic implants may have low fatigue strength relative to components made of the composite material. Metal components of orthopedic implants may interfere with some imaging procedures (e.g., such as X-Ray, CT, etc.). Metal components of orthopedic implants may prevent a subject implanted with such metal components from undergoing some medical procedures (e.g., such as MRI, radiation therapy, etc.).

### SUMMARY OF THE INVENTION

Some embodiments of the present invention provide a composite material device for connecting an orthopedic rod to a pedicle screw, wherein the device includes: a housing having a substantially annular body and including: two opposite concave indents at a proximal end of the housing and in a longitudinal direction along the housing, the concave indents being structured to receive an orthopedic rod; and an internal thread on an internal surface of a proximal portion of the housing; an adapter structured to lock at least a portion of a pedicle screw head within a distal portion of the housing; an internal bolt including an external thread configured for mating with the internal thread of the housing, wherein the internal bolt is structured to be screwed into the proximal portion of the housing so as to lock the orthopedic rod between the adapter and the internal bolt; and an external fastener structured to tightly surround at least a part of the proximal portion of the housing.

In some embodiments, the adapter includes two longitudinal halves structured to embrace and lock at least a portion of the pedicle screw head therebetween and to be tightly inserted into the distal portion of the housing when embracing the pedicle screw head.

In some embodiments, the device further includes a spacer structured to be inserted into the housing between the adapter and the internal bolt so that, when the internal bolt is being screwed into the proximal portion of the housing, the internal bolt presses the spacer against the orthopedic rod between the adapter and the spacer.

In some embodiments, the housing includes an external thread, and the external fastener has a substantially annular body and includes an internal thread on an inner lateral surface thereof, and the internal thread of the external fastener mates with the external thread of the housing.

In some embodiments, one thread of the internal thread and the external thread of the housing is a right-hand thread, and another thread of the internal thread and the external thread of the housing is a left-hand thread.

In some embodiments, at least one of the external thread of the housing, the internal thread of the housing, the external thread of the internal bolt and the internal thread of the external fastener is coated with a coating material that includes polyether ether ketone (PEEK) or metal.

In some embodiments, a thickness of a layer of the coating material is between 2.5-40 μm.

In some embodiments, the external fastener has a substantially annular body and includes two opposite protrusions protruding inwardly from an inner lateral surface of the external fastener, wherein the protrusions of the external fastener are structured to be tightly inserted into the first and the second concave indents of the housing when the external fastener is connected to the proximal portion of the housing.

In some embodiments, the housing includes an internal composite material layer and an external composite material layer, wherein fibers of the composite material are arranged in different directions in the internal composite material layer and in the external composite material layer.

In some embodiments, in one layer of the internal composite material layer and the external composite material layer, fibers of the composite material are arranged in a tangential direction, and in another layer of the internal composite material layer and the external composite material layer, fibers of the composite material are arranged in an axial direction.

In some embodiments, fibers of the composite material are helically arranged at least along the length of the housing.

In some embodiments, an internal surface of the distal portion of the housing tapers in the longitudinal direction of the housing, a curved longitudinal surface of each of the longitudinal halves of the adapter tapers in a longitudinal direction of the respective halve, and the tapering of the internal surface of the distal portion of the housing mates with the tapering of curved longitudinal surfaces of the longitudinal halves.

In some embodiments, each of the longitudinal halves of the adapter includes a recess on the flat longitudinal surface thereof, the recess extending from a distal end towards a proximal end in the longitudinal direction along a portion of a length of the respective longitudinal halve, and the longitudinal recesses of the longitudinal halves are structured to receive and lock at least a portion of the pedicle screw head when the longitudinal halves are positioned in proximity to each other and are aligned with respect to their flat longitudinal surfaces.

In some embodiments, the housing includes two opposite slot apertures on its distal portion, and each of the longitudinal halves of the adapter includes at least one protrusion on its curved longitudinal surface, wherein the at least one protrusion is structured and positioned to be slidably and tightly insertable into one of the slot apertures on the distal portion of the housing.

In some embodiments, the slot apertures are elongated in the longitudinal direction along the housing, and the at least one protrusion of each of the longitudinal halves of the adapter are elongated in a longitudinal direction of the respective longitudinal halve.

In some embodiments, each of the longitudinal halves of the adapter includes a concave indent at its proximal end and in the longitudinal direction of the respective longitudinal halve, wherein the concave indent extends along a transverse dimension of the proximal end of the respective longitudinal halve and is structured to receive a portion of the orthopedic rod.

In some embodiments, the spacer is substantially flat and has an upper flat surface and a bottom flat surface, wherein the spacer includes a concave indent on the bottom flat surface thereof, and the concave indent extends along a longitudinal dimension of the spacer and is structured to receive a portion of the orthopedic rod.

In some embodiments, the spacer includes a central circular portion structured to be inserted into an interior of the housing, and two opposite radial protrusion radially protruding from opposite sides of the central circular portion, wherein the radial protrusions are structured to be inserted into the first and second concave idents of the housing.

In some embodiments, each of the radial protrusions includes two lateral portions laterally protruding from opposite lateral sides of a distal end of the respective radial protrusion.

In some embodiments, the upper flat surface of the spacer is coated with a coating material in at least one of the central circular portion and the distal ends of the radial protrusions of the spacer, wherein the coating material includes PEEK or metal.

In some embodiments, a thickness of a layer of the coating material is between 2.5-40 μm.

In some embodiments, the concave indent of each of the longitudinal halves of the adapter includes two opposite sloped surfaces connected at their distal ends to opposite proximal ends of a concave arc, and the spacer includes two opposite spacer protrusions protruding from the central circular portion of the bottom flat surface of the spacer, wherein external surfaces of the spacer protrusions are sloped and mate with the sloped surfaces of concave indents of the longitudinal halves of the adapter.

In some embodiments, the spacer is connected at its center point to a distal end of the internal bolt and is rotatable with respect to the internal bolt about a central longitudinal axis of the internal bolt.

Some embodiments of the present invention provide a composite material pedicle screw sub-assembly, wherein the pedicle screw sub-assembly includes: a pedicle screw having a head and a central longitudinal axis; a housing having a substantially annular body, a proximal portion, a distal portion and a central longitudinal axis; an adapter including two longitudinal halves positioned within the distal portion of the housing, wherein the longitudinal halves embrace and lock a portion of the pedicle screw head such that the central longitudinal axis of the pedicle screw coincides with the central longitudinal axis of the housing; and wherein the longitudinal halves of the adapter are prepressed in a longitudinal direction of the housing so as to maintain the coincidence of the central longitudinal axis of the pedicle screw with the central longitudinal axis of the housing.

In some embodiments, the housing includes two opposite slot apertures on its distal portion, and each of the longitudinal halves of the adapter includes at least one protrusion on its curved longitudinal surface, wherein the at least one protrusion is structured and positioned to be slidably and tightly insertable into one of the slot apertures on the distal portion of the housing.

In some embodiments, the slot apertures are elongated in the longitudinal direction along the housing, and the at least one protrusion of each of the longitudinal halves of the adapter are elongated in a longitudinal direction of the respective longitudinal halve.

In some embodiments, a thread of the pedicle screw is coated with a coating material includes PEEK or metal.

In some embodiments, a thickness of a layer of the coating material is between 2.5-40 μm.

Some embodiments of the present invention provide a surgical tool removably connectable to a composite material pedicle screw sub-assembly having a pedicle screw, a pedicle screw sub-assembly housing and a pedicle screw sub-assembly adapter positioned and prepressed within a distal portion of the pedicle screw sub-assembly housing and locking a portion of a pedicle screw head, wherein the surgical tool includes: a tool rod includes a proximal end removably connectable to a screwing tool handle, and a distal end having a shape and size that mate with a shape and size of a proximal end of the pedicle screw sub-assembly adapter; a tool tube having a substantially annular body and accommodating at least a portion of the tool rod, the tool tube including an external thread on an external lateral surface of its distal end, wherein the external thread of the tool tube mates with an internal thread of a proximal portion of the pedicle screw sub-assembly housing; and a tool handle having a substantially annular body, wherein the tool tube accommodates at least a portion of the tool rod and is coupled to the tool tube so that the tool handle and the tool rod are rotatable with respect to the tool rod about a tool central longitudinal axis.

In some embodiments, the tool tube includes an envelope that surrounds the distal end of the tool tube, wherein the envelope is structured to receive the proximal portion of the pedicle screw sub-assembly housing when the tool tube is being screwed into the proximal portion of the pedicle screw sub-assembly housing.

In some embodiments, the surgical tool includes a torque limiter structured to decouple the tool tube from the tool handle so as to terminate screwing of the tool handle into the pedicle screw sub-assembly housing when a torque being applied on the tool handle exceeds a preset torque threshold, and to maintain coupling of the tool tube with the tool handle during unscrewing of the tool handle from the pedicle screw sub-assembly housing independent of the torque being applied on the tool handle.

In some embodiments, the torque limiter is mounted within the tool handle.

In some embodiments, the torque limiter includes: a spool housing mounted within and rigidly connected to the tool handle; a spring-loaded spool mounted and longitudinally movable within the spool housing; multiple detents between the spool housing and the tool handle; and multiple balls each being held within one of the detents by the spring-loaded spool so as to couple the tool handle to the spool housing and thus to the tool tube; wherein each of the detents is asymmetric with respect to a radial direction of the tool tube handle.

In some embodiments, each of the detents includes: an arc-like surface shaped and sized to accommodate at least a half of a diameter of a ball of the multiple balls; and a sloped surface connected to the arc-like surface.

Some embodiments of the present invention provide a device for applying a counter torque, the device includes: a substantially annular body having a proximal end, a distal end and an interior structured to receive a screwing tool; two opposite concave indents at the distal end in a longitudinal direction of the annular body, the concave indents are structured to receive an orthopedic rod; and a handle connected to the annular body and perpendicular to the longitudinal direction of the annular body.

In some embodiments, the handle is connected to an external lateral surface of the annular body at the proximal end of the annular body.

In some embodiments, the handle is removably connectable to the annular body.

Some embodiments of the present invention provided a screwing tool for screwing a screwable external fastener to a device having a housing and an internal bolt screwed into the housing, wherein the screwing tool includes: a rod having a proximal end and a distal end, wherein the proximal end is structured to be connected to a screwing tool handle; a gripper connected to the distal end of the rod and structured to grip the external fastener; and a spring-loaded pin connected to the distal end of the rod along a central longitudinal axis of the rod using a spring, wherein the spring-loaded pin centralizes the external fastener with respect to the internal bolt when the external fastener is being gripped by the gripper and is being screwed onto the housing of the device.

Some embodiments of the present invention provide a screwing tool for simultaneous screwing and tightening of an internal bolt and an external fastener screwable to a housing of a device, wherein the screwing tool includes: an input rod having a proximal end and a distal end, the proximal end being removably connectable to a screwing tool handle; a transmission coupled to the distal end of the input rod; an internal bolt rod coupled at its proximal end to the transmission and having a distal end that is removably connectable to the internal bolt of the device; and an external fastener tube accommodating the internal bolt rod such that the internal bolt rod and the external fastener tube are rotatable with respect to each other about their respective central longitudinal axes, wherein the external fastener tube is coupled at its proximal end to the transmission and is removably connectable at its distal end to the external fastener of the device; wherein the transmission is structured to rotate the internal bolt rod and the external fastener tube about their respective central longitudinal axes in response to rotation of the input rod about a central longitudinal axis of the input rod.

In some embodiments, the transmission is structured to rotate the internal bolt rod and the external fastener tube about their respective central longitudinal axes in opposite directions with respect to each other in response to one-directional rotation of the input rod about the central longitudinal axis of the input rod.

In some embodiments, the transmission is structured to rotate the internal bolt rod and the external fastener tube about their respective central longitudinal axes in the same direction with respect to each other in response to rotation of the input rod about the central longitudinal axis of the input rod.

In some embodiments, a transmission ratio of the transmission and directions of rotations of the internal bolt rod and of the external fastener tube at an output of the transmission are preset based on known screwing directions and known tightening torque values of the internal bolt and the external fastener of the device.

In some embodiments, the transmission includes a gear assembly.

In some embodiments, the transmission includes a planetary gear assembly.

In some embodiments, the transmission includes: a sun gear rigidly connected to the distal end of the input rod so that a sun gear axis coincides with the input rod central longitudinal axis; a first planet gear meshed with the sun gear, the first planet gear being rotatable about a first planet gear axis that is substantially parallel to the sub gear axis; a second planet gear rigidly connected to the first planet gear on the first planet gear axis such that the second planet gear and the first planet gear are rotatable about the first planet gear axis; a third planet gear meshed with the second planet gear, the third planet gear being rotatable about a third planet gear axis that is substantially parallel to the sun gear axis; a ring gear meshed with the third planet gear and rigidly connected to the proximal end to the internal bolt rod such that a ring gear axis coincides with the internal bolt rod central longitudinal axis; and a carrier rotatable about the sun gear axis and rigidly connected to the external fastener tube so as to rotate the external fastener tube about the external fastener central longitudinal axis when the carrier rotates about the sun gear axis, wherein the first and second planet gears are rotatably coupled to the carrier on the first planet gear axis, and wherein the third planet gear is rotatably coupled to the carrier on the third planet gear axis.

In some embodiments, the transmission includes: a sun gear rigidly connected to the distal end of the input rod, so that a sun gear axis coincides with the input rod central longitudinal axis; a first planet gear meshed with the sun gear, the first planet gear being rotatable about a first planet gear axis that is substantially parallel to the sub gear axis; a second planet gear rigidly connected to the first planet gear on the first planet gear axis such that the second planet gear and the first planet gear are rotatable about the first planet gear axis; a ring gear meshed with the second planet gear and rigidly connected to the proximal end to the internal bolt rod such that a ring gear axis coincides with the internal bolt rod central longitudinal axis; and a carrier rotatable about the sun gear axis and rigidly connected to the external fastener tube so as to rotate the external fastener tube about the external fastener central longitudinal axis when the carrier rotates about the sun gear axis, wherein the first and second planet gears are rotatably coupled to the carrier on the first planet gear axis.

In some embodiments, a number of teeth on each of gears in the transmission is preset based on a desired torque transmission ratio of the transmission.

Some embodiments of the present invention provide a composite material device for connecting an orthopedic rod to a pedicle screw, the device may include: a housing having a substantially annular body and comprising: two opposite concave indents at a proximal end of the housing and in a longitudinal direction along the housing, the concave indents being configured to receive an orthopedic rod; and an internal thread on an internal surface of a proximal portion of the housing; an adapter configured to lock at least a portion of a pedicle screw head within a distal portion of the housing; and an internal bolt comprising an external thread configured to mate with the internal thread of the housing, the internal bolt being configured to be screwed into the proximal portion of the housing so as to lock the orthopedic rod between the adapter and the internal bolt; wherein the housing, the adapter and the internal bolt comprise composite material.

In some embodiments, the adapter comprises two longitudinal halves being configured to embrace and lock at least a portion of the pedicle screw head therebetween and to be tightly inserted into the distal portion of the housing when embracing the pedicle screw head.

In some embodiments, each of the longitudinal halves of the adapter comprises a flat longitudinal surface and a curved longitudinal surface.

In some embodiments, the flat longitudinal surface of each of the longitudinal halves comprises a recess extending from a distal end towards a proximal end in a longitudinal direction along a portion of a length of the respective longitudinal half.

In some embodiments, the longitudinal recesses of the longitudinal halves are configured to receive and lock at least a portion of the pedicle screw head when the longitudinal halves are in a proximity to each other and are aligned with respect to their flat longitudinal surfaces.

In some embodiments, the curved longitudinal surface of each of the longitudinal halves tapers in the longitudinal direction of the respective longitudinal halve.

In some embodiments, an internal surface of the distal portion of the housing tapers in the longitudinal direction of the housing.

In some embodiments, the tapering of the internal surface of the distal portion of the housing mates with the tapering of curved longitudinal surfaces of the longitudinal halves.

In some embodiments, each of the longitudinal halves of the adapter comprises a concave indent extending in a transverse dimension at a proximal end of the respective longitudinal halve and being configured to receive a portion of the orthopedic rod.

In some embodiments, the housing and the adapter are configured to at least limit rotational and axial motion of the adapter with respect to the housing when the adapter is received within the housing.

In some embodiments, the device further includes two restriction pins that are configured to at least limit rotational and axial motion of the adapter with respect to the housing when the adapter is received within the housing.

In some embodiments, the housing comprises two opposing housing pin openings made through an external lateral surface of the housing.

In some embodiments, each of the longitudinal halves of the adapter comprises an adapter groove configured to receive a portion of one of the restriction pins through one of the housing pin openings.

In some embodiments, each of the restriction pins is connectable within one of the housing openings.

In some embodiments, the device further includes a spacer configured to be inserted into the housing between the adapter and the internal bolt so that, when the internal bolt is screwed into the proximal portion of the housing, the internal bolt presses the spacer against the orthopedic rod between the adapter and the spacer.

In some embodiments, the spacer is substantially flat and has an upper flat surface and a bottom flat surface, wherein the spacer comprises a concave indent on the bottom flat surface thereof, and wherein the concave indent extends along a longitudinal dimension of the spacer and is configured to receive a portion of the orthopedic rod.

In some embodiments, the spacer includes: a central circular portion configured to be tightly inserted into an interior of the housing; and two opposite radial protrusions radially protruding from opposing sides of the central circular portion, wherein the radial protrusions are configured to be tightly inserted into the first concave indent and the second concave ident of the housing.

In some embodiments, each of the radial protrusions comprises a sloped surface at its respective end.

In some embodiments, the upper flat surface of the spacer is coated with a coating material at least in the central circular portion of the spacer, wherein the coating material comprises PEEK or metal.

In some embodiments, a thickness of a layer of the coating material is between 2.5-40 μm.

In some embodiments, the spacer is connected at its center point to a distal end of the internal bolt.

In some embodiments, the spacer is rotatable with respect to the internal bolt about a central longitudinal axis of the internal bolt.

In some embodiments, at least either the internal thread of the housing or the external thread of the internal bolt is coated with a coating material comprising polyether ether ketone (PEEK) or metal.

In some embodiments, a thickness of a layer of the coating material is between 2.5-40 μm.

In some embodiments, the internal thread of the housing and the external thread of the internal bolt are tilted relative to a plane that is perpendicular to the longitudinal direction of the housing and mating each other.

In some embodiments, the housing comprises composite material fibers being arranged along at least a portion of the housing in at least one of: an axial direction of the housing, a radial direction of the housing, a clockwise helical direction along the housing, a counterclockwise helical direction along the housing, or any combination thereof.

In some embodiments, the housing comprises two or more layers of composite material fibers, wherein the composite material fibers in at least a portion of the two or more layers are arranged in different direction with respect to each other.

In some embodiments, the internal bolt comprises composite material fibers being arranged at along at least a portion of the internal bolt in at least one of: an axial direction of the internal bolt, a radial direction of the internal bolt, a clockwise helical direction along the internal bolt, a counterclockwise helical direction along the internal bolt, or any combination thereof.

In some embodiments, the internal bolt comprises two or more layers of composite material fibers, wherein the composite material fibers in at least a portion of the two or more layers are arranged in different direction with respect to each other.

Some embodiments of the present invention may provide a composite material pedicle screw sub-assembly, the pedicle screw sub-assembly may include: a pedicle screw having a head and a central longitudinal axis; a housing having a substantially annular body, a proximal portion, a distal portion and a central longitudinal axis; an adapter comprising two longitudinal halves positioned within the distal portion of the housing, wherein the longitudinal halves are configured to embrace and lock a portion of the pedicle screw head such that the central longitudinal axis of the pedicle screw coincides with the central longitudinal axis of the housing; wherein the longitudinal halves of the adapter are prepressed in a longitudinal direction of the housing so as to maintain the coincidence of the central longitudinal axis of the pedicle screw with the central longitudinal axis of the housing.

In some embodiments, the sub-assembly further includes two restriction pins being configured to at least limit rotation of the longitudinal halves of the adapter with respect to the housing.

In some embodiments, the housing comprises two opposing housing pin openings made through an external lateral surface of the housing.

In some embodiments, each of the longitudinal halves of the adapter comprises an adapter groove.

In some embodiments, a first portion of each of the restriction pins is connected within one of the housing openings and a second portion of the respective restriction pin is received within the adapter groove of one of the longitudinal halves.

Some embodiments of the present invention provide a composite material pedicle screw implant assembly including: an orthopedic rod; a pedicle screw; and a device for connecting the orthopedic rod to the pedicle screw, the device including: a housing having a substantially annular body and including: two opposite concave indents at a proximal end of the housing and in a longitudinal direction along the housing, the concave indents being configured to receive an orthopedic rod; and an internal thread on an internal surface of a proximal portion of the housing; an adapter configured to lock at least a portion of a pedicle screw head within a distal portion of the housing; and an internal bolt including an external thread configured to mate with the internal thread of the housing, the internal bolt being configured to be screwed into the proximal portion of the housing so as to lock the orthopedic rod between the adapter and the internal bolt; wherein the orthopedic rod, the pedicle screw, the housing, the adapter and the internal bolt include composite material.

In some embodiments, the adapter includes two longitudinal halves being configured to embrace and lock at least a portion of the pedicle screw head therebetween and to be tightly inserted into the distal portion of the housing when embracing the pedicle screw head.

In some embodiments, each of the longitudinal halves of the adapter includes a flat longitudinal surface and a curved longitudinal surface.

In some embodiments, the flat longitudinal surface of each of the longitudinal halves includes a recess extending from a distal end towards a proximal end in a longitudinal direction along a portion of a length of the respective longitudinal half.

In some embodiments, the longitudinal recesses of the longitudinal halves are configured to receive and lock at least a portion of the pedicle screw head when the longitudinal halves are in a proximity to each other and are aligned with respect to their flat longitudinal surfaces.

In some embodiments, the curved longitudinal surface of each of the longitudinal halves tapers in the longitudinal direction of the respective longitudinal half.

In some embodiments, an internal surface of the distal portion of the housing tapers in the longitudinal direction of the housing.

In some embodiments, the tapering of the internal surface of the distal portion of the housing mates with the tapering of curved longitudinal surfaces of the longitudinal halves.

In some embodiments, each of the longitudinal halves of the adapter includes a concave indent extending in a transverse dimension at a proximal end of the respective longitudinal halve and being configured to receive a portion of the orthopedic rod.

In some embodiments, the housing and the adapter are configured to at least limit rotational and axial motion of the adapter with respect to the housing when the adapter is received within the housing.

In some embodiments, the assembly further includes two restriction pins that are configured to at least limit rotational and axial motion of the adapter with respect to the housing when the adapter is received within the housing.

In some embodiments, the housing includes two opposing housing pin openings made through an external lateral surface of the housing.

In some embodiments, each of the longitudinal halves of the adapter includes an adapter groove configured to receive a portion of one of the restriction pins through one of the housing pin openings.

In some embodiments, each of the restriction pins is connectable within one of the housing openings.

In some embodiments, the assembly further includes a spacer configured to be inserted into the housing between the adapter and the internal bolt so that, when the internal bolt is screwed into the proximal portion of the housing, the internal bolt presses the spacer against the orthopedic rod between the adapter and the spacer.

In some embodiments, the spacer is substantially flat and has an upper flat surface and a bottom flat surface, wherein the spacer includes a concave indent on the bottom flat surface thereof, and wherein the concave indent extends along a longitudinal dimension of the spacer and is configured to receive a portion of the orthopedic rod.

In some embodiments, the spacer includes a central circular portion configured to be tightly inserted into an interior of the housing.

In some embodiments, the spacer includes two opposite radial protrusions radially protruding from opposing sides of the central circular portion, wherein the radial protrusions are configured to be tightly inserted into the first concave indent and the second concave ident of the housing.

In some embodiments, each of the radial protrusions includes a sloped surface at its respective end.

In some embodiments, the upper flat surface of the spacer is coated with a coating material at least in the central circular portion of the spacer, wherein the coating material includes PEEK or metal.

In some embodiments, the spacer is connected at its center point to a distal end of the internal bolt and wherein is rotatable with respect to the internal bolt about a central longitudinal axis of the internal bolt.

In some embodiments, at least either the internal thread of the housing or the external thread of the internal bolt is coated with a coating material including polyether ether ketone (PEEK) or metal.

In some embodiments, the internal thread of the housing and the external thread of the internal bolt are tilted relative to a plane that is perpendicular to the longitudinal direction of the housing and mating each other.

In some embodiments, the housing includes composite material fibers being arranged along at least a portion of the housing in at least one of: an axial direction of the housing, a radial direction of the housing, a clockwise helical direction along the housing, a counterclockwise helical direction along the housing, or any combination thereof.

In some embodiments, the housing includes two or more layers of composite material fibers, wherein the composite material fibers in at least a portion of the two or more layers are arranged in different direction with respect to each other.

In some embodiments, the internal bolt includes composite material fibers being arranged at along at least a portion of the internal bolt in at least one of: an axial direction of the internal bolt, a radial direction of the internal bolt, a clockwise helical direction along the internal bolt, a counterclockwise helical direction along the internal bolt, or any combination thereof.

In some embodiments, the internal bolt includes two or more layers of composite material fibers, wherein the composite material fibers in at least a portion of the two or more layers are arranged in different direction with respect to each other.

### BRIEF DESCRIPTION OF THE DRAWINGS

For a better understanding of embodiments of the invention and to show how the same can be carried into effect, reference will now be made, purely by way of example, to the accompanying drawings in which like numerals designate corresponding elements or sections throughout.

In the accompanying drawings:

FIGS. 1A and 1B are schematic illustrations of pedicle screw implant assembly, according to some embodiments of the invention;

FIGS. 2A, 2B, 2C and 2D are schematic illustrations of a device for connecting an orthopedic rod to a pedicle screw, according to some embodiments of the invention;

FIGS. 2E, 2F and 2G are schematic illustrations of a housing of a device for connecting an orthopedic rod to a pedicle screw, according to some embodiments of the invention;

FIG. 2H is a schematic illustration of an adapter of a device for connecting an orthopedic rod to a pedicle screw, according to some embodiments of the invention;

FIGS. 2I, 2J and 2K are schematic illustrations of a longitudinal half of an adapter of a device for connecting an orthopedic rod to a pedicle screw, according to some embodiments of the invention;

FIGS. 2L, 2M, 2N and 2O are schematic illustrations of an internal bolt and a spacer of a device for connecting an orthopedic rod to a pedicle screw, according to some embodiments of the invention;

FIGS. 2P, 2Q and 2R are schematic illustrations of an external fastener, according to some embodiments of the invention;

FIGS. 3A, 3B and 3C are schematic illustrations of a pedicle screw sub-assembly including a housing, an adapter and a pedicle screw, according to some embodiments of the invention;

FIG. 4A is a schematic illustration of a device for connecting an orthopedic rod to a pedicle screw, and of orthopedic rod and pedicle screw, according to some embodiments of the invention;

FIG. 4B is a schematic illustration of an adapter of a device for connecting an orthopedic rod to a pedicle screw, according to some embodiments of the invention;

FIG. 4C is a schematic illustration of a longitudinal halve of an adapter of a device for connecting an orthopedic rod to a pedicle screw, according to some embodiments of the invention;

FIGS. 4D and 4E are schematic illustrations of a spacer of a device for connecting an orthopedic rod to a pedicle screw, according to some embodiments of the invention;

FIGS. 4F, 4G and 4H are schematic illustrations of a device for connecting an orthopedic rod to a pedicle screw, and of orthopedic rod and pedicle screw, according to some embodiments of the invention;

FIG. 5A is schematic illustrations of a surgical tool, according to some embodiments of the invention.

FIG. 5B is a schematic illustration of a surgical tool connected to a pedicle screw sub-assembly, according to some embodiments of the invention;

FIGS. 5C and 5D are schematic illustrations of a section A-A view of FIG. 4F showing a distal end of a surgical tool connected to a pedicle screw sub-assembly, according to some embodiments of the invention;

FIGS. 5E and 5F are schematic illustrations of a torque limiter of a surgical tool, according to some embodiments of the invention;

FIGS. 6A, 6B and 6C are schematic illustrations of a device for applying a counter torque, according to some embodiments of the invention;

FIG. 7A is a schematic illustration of a screwing tool for screwing an internal bolt of a device for connecting an orthopedic rod to a pedicle screw, according to some embodiment of the invention;

FIG. 7B is a schematic illustration of a screwing tool for screwing an internal bolt of a device for connecting an orthopedic rod to a pedicle screw, positioned within device for applying a counter torque, wherein the device for applying a counter torque is connected to an orthopedic rod, according to some embodiments of the invention;

FIGS. 8A, 8B and 8C are schematic illustrations of an assembly including a screwing tool for screwing a screwable external fastener of a device including a housing and an internal bolt screwed into housing, a device for holding a screwable tool, an orthopedic rod and a pedicle screw, according to some embodiments of the invention; and

FIGS. 9A, 9B and 9C are schematic illustrations of a screwing tool for simultaneous screwing/tightening of an internal bolt and an external fastener screwable to a housing of a device, according to some embodiments of the invention;

FIG. 9D is a schematic illustration of a section A-A view of FIG. 9A showing a screwing tool for simultaneous screwing/tightening of an internal bolt and an external fastener screwable in opposite screwing directions, including a schematic section A-A view of a transmission of screwing tool, according to some embodiments of the invention;

FIGS. 9E and 9F are schematic illustrations of a transmission of a screwing tool for simultaneous screwing/tightening of an internal bolt and an external fastener screwable in opposite screwing directions, according to some embodiments of the invention;

FIGS. 10A and 10B are schematic illustrations of a device for connecting an orthopedic rod to a pedicle screw, according to some embodiments of the invention;

FIGS. 10C, 10D and 10E are schematic illustrations of different configurations of an adapter of a device for connecting an orthopedic rod to a pedicle screw, according to some embodiments of the invention;

FIGS. 11A and 11B are schematic illustrations of a pedicle screw implant assembly, according to some embodiments of the invention;

FIGS. 12A, 12B, 12C and 12D are schematic illustrations of a device for connecting an orthopedic rod to a pedicle screw, according to some embodiments of the invention;

FIGS. 12E, 12F and 12G are schematic illustrations of a housing of the device for connecting an orthopedic rod to a pedicle screw, according to some embodiments of the invention;

FIG. 12H is a schematic illustration of an adapter of the device for connecting an orthopedic rod to a pedicle screw, according to some embodiments of the invention;

FIGS. 12I, 12J and 12K are schematic illustrations of a longitudinal half of an adapter of the device for connecting an orthopedic rod to a pedicle screw, according to some embodiments of the invention;

FIGS. 12L, 12M, 12N and 12O are schematic illustrations of an internal bolt and a spacer of the device for connecting an orthopedic rod to a pedicle screw, according to some embodiments of the invention;

FIGS. 12P, 12Q, 12R and 12S are schematic illustrations of the spacer of the device for connecting an orthopedic rod to a pedicle screw, according to some embodiments of the invention;

FIGS. 12T, 12U and 12V are schematic illustrations of the internal bolt and a spacer of device for connecting an orthopedic rod to a pedicle screw, according to some embodiments of the invention;

FIGS. 13A, 13B and 13C are schematic illustrations of a pedicle screw sub-assembly including a housing, an adapter, restriction pins and a pedicle screw, according to some embodiments of the invention;

FIGS. 14A, 14B, 14C, 14D and 14E are schematic illustrations of a tool for screwing pedicle screw sub-assembly into a vertebra of a subject, according to some embodiments of the invention;

FIGS. 14F, 14G, 14H, 14I and 14J are schematic illustrations of tool including a ring member and a spring, according to some embodiments of the invention;

FIGS. 15A, 15B,15C and 15D, which are schematic illustrations of a tool for loosening of a coupling of housing/adapter with pedicle screw of pedicle screw sub-assembly, according to some embodiments of the invention;

FIGS. 15E, 15F, 15G and 15H are schematic illustrations of the coupling loosening tool with a gripper including an internal member, according to some embodiments of the invention;

FIGS. 16A, 16B, 16C, 16D, 16E and 16F are schematic illustrations of a tool for positioning and holding an orthopedic rod into a housing of pedicle screw sub-assembly, according to some embodiments of the invention;

FIGS. 16G, 16H and 161 are schematic illustrations of a gripper of the orthopedic rod positioning and holding tool, according to some embodiments of the invention;

FIGS. 17A, 17B, 17C and 17D are schematic illustrations of a tool for screwing an internal bolt into a housing of a pedicle screw assembly, according to some embodiments of the invention;

FIGS. 18A, 18B, 18C, 18D and 18E are schematic illustrations of a tool for screwing an internal bolt into a housing of a pedicle screw assembly, according to some embodiments of the invention;

FIGS. 19A, 19B, 19C, 19D, 19E, 19F and 19G are schematic illustrations of a tool for applying a counter torque, according to some embodiments of the invention; and

FIGS. 20A, 20B, 20C, 20D and 20E are schematic illustrations of a tool for screwing an internal bolt into housing of pedicle screw sub-assembly, according to some embodiments of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention can be practiced without the specific details presented herein. Furthermore, well known features can have been omitted or simplified in order not to obscure the present invention. With specific reference to the drawings, it is stressed that the particulars shown are by way of example and for purposes of illustrative discussion of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention can be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that can be practiced or carried out in various ways as well as to combinations of the disclosed embodiments. Also, it is to be understood that the phraseology and terminology employed herein is for the purpose of description and should not be regarded as limiting.

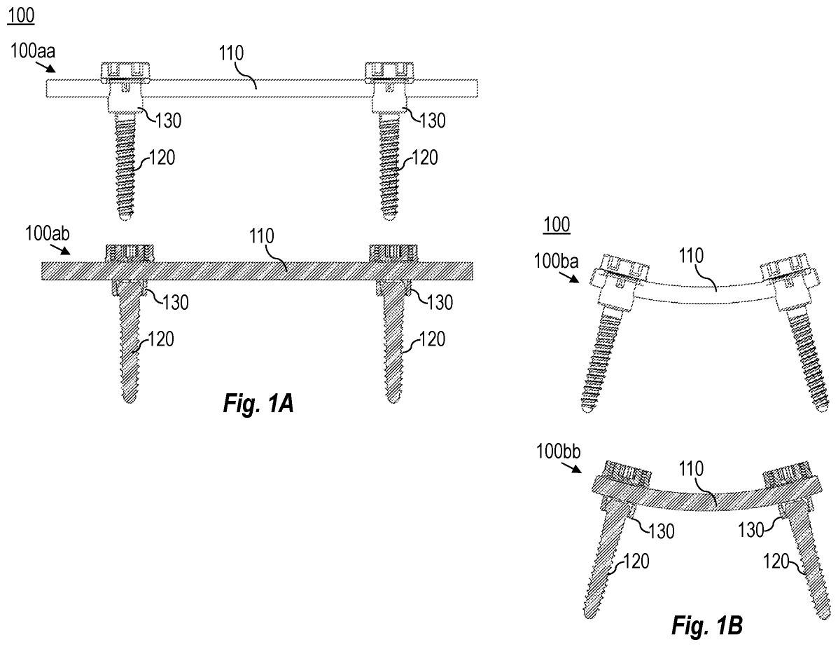

Reference is now made to FIGS. 1A and 1B, which are schematic illustrations of pedicle screw implant assembly 100, according to some embodiments of the invention.

Illustrations 100aa and 100ba in FIGS. 1A and 1B, respectively, show side views of pedicle screw implant assembly 100. Illustrations 100ab and 100bb in FIGS. 1A and 1B, respectively, show section views of pedicle screw implant assembly 100.

Pedicle screw implant assembly 100 may include an orthopedic rod 110, a pedicle screw 120 and a device 130 for connecting orthopedic rod 110 to pedicle screw 120.

In some embodiments, orthopedic rod 110 is straight (e.g., as shown in FIG. 1A). In some embodiments, orthopedic rod 110 is bent (e.g., as shown in FIG. 1B).

Orthopedic rod 110, pedicle screw 120 and device 130 according to some embodiments of the present invention are made of a composite material. In some embodiments, the composite material includes polyether ether ketone (PEEK). In some embodiments, the composite material includes carbon fibers. In some embodiments, the composite material includes PEEK and carbon fibers. For example, the composite material may include 40% PEEK and 60% carbon fibers.

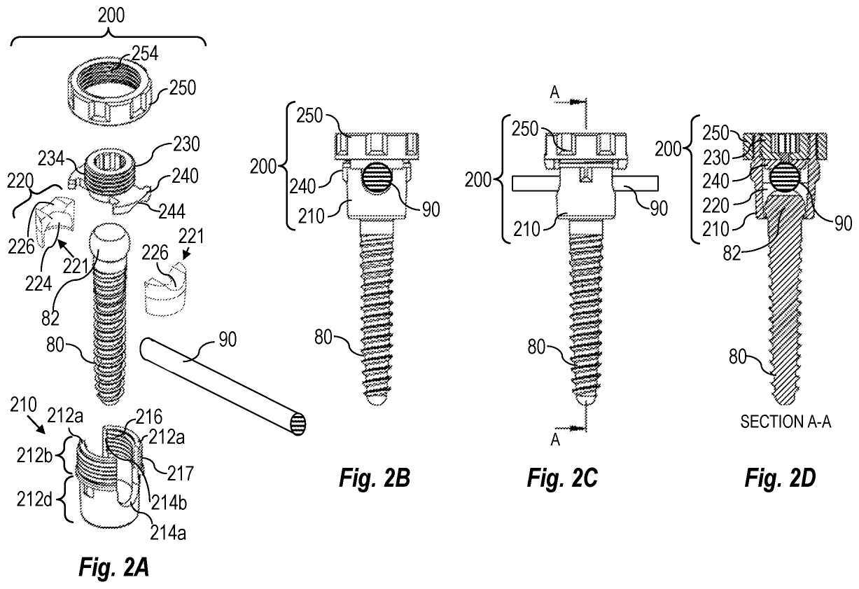

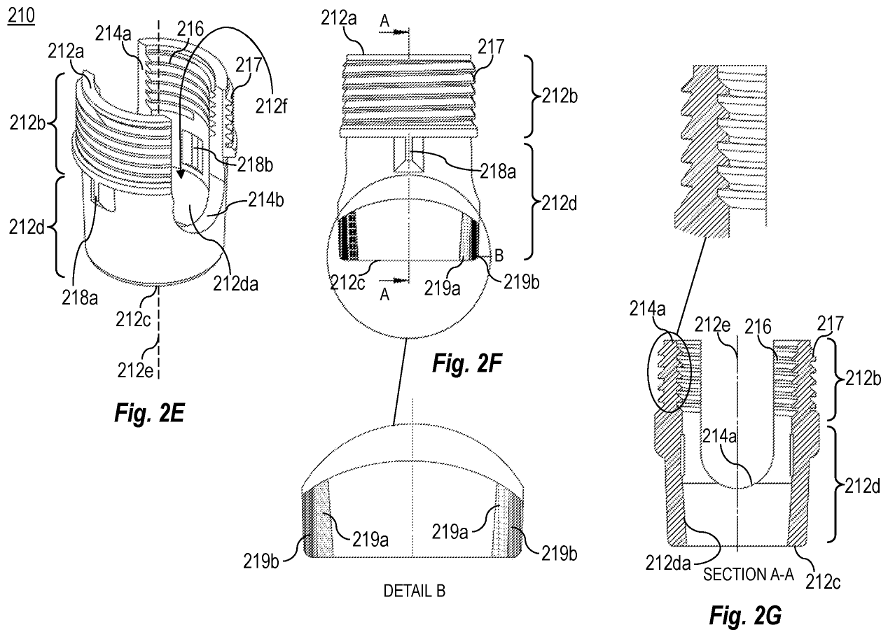

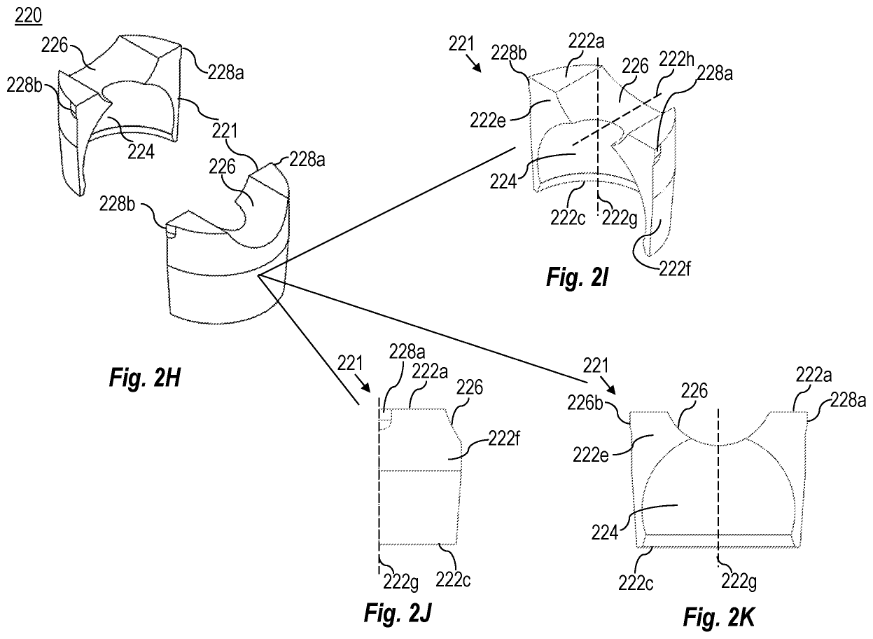

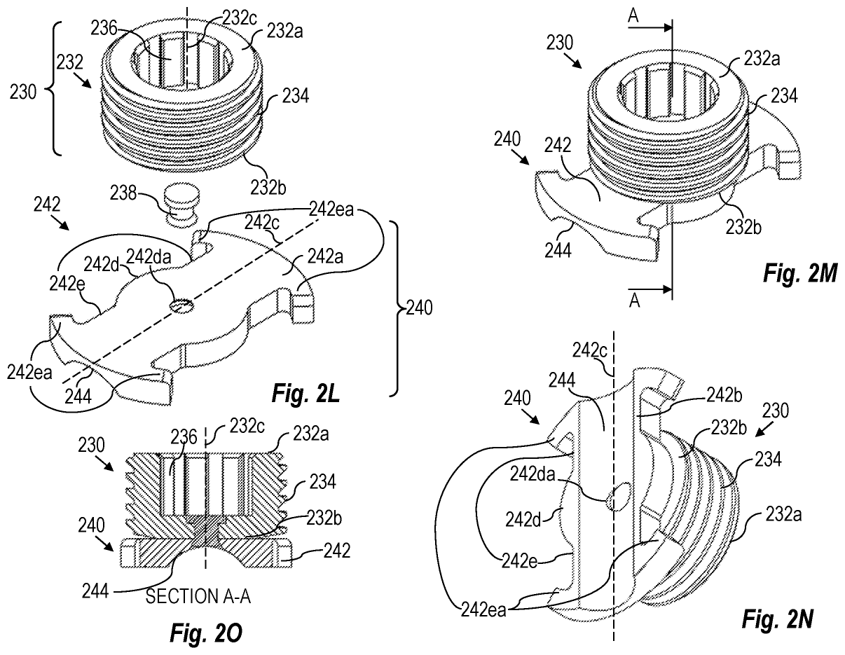

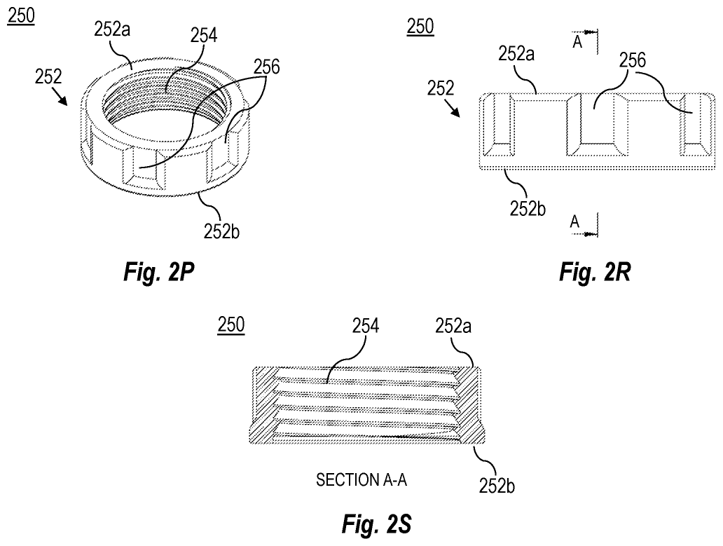

Reference is now made to FIGS. 2A, 2B, 2C and 2D, which are schematic illustrations of a device 200 for connecting an orthopedic rod 90 to a pedicle screw 80, according to some embodiments of the invention.

FIG. 2A shows a perspective view of a disassembled device 200 and of pedicle screw 80. FIGS. 2B and 2C show different side views of assembled device 200 connected to pedicle screw 80 and orthopedic rod 90. FIG. 2D shows a section A-A view of assembled device 200 connected to orthopedic rod 90 and pedicle screw 80.

According to some embodiments of the invention, device 200 for connecting an orthopedic rod 90 to a pedicle screw 80 includes a housing 210, an adapter 220, an internal bolt 230, a spacer 240 and an external fastener 250. Housing 210, adapter 220, internal bolt 230, spacer 240 and external fastener 250 according to some embodiments of the present invention are made of the composite material.

Housing 210 may have a substantially annular body. Housing 210 may include two opposite concave indents 214a, 214b at its proximal end 212a in a longitudinal direction of housing 210. Concave indents 214a, 214b may be shaped and sized to receive orthopedic rod 90. Housing 210 may include an internal thread 216 on an internal surface of a proximal portion 212b of housing 210. In embodiments shown in FIGS. 2A, 2B, 2C and 2D, housing 210 includes an external thread 217 on an external surface of proximal portion 212b of housing 210. Embodiments of housing 210 are described below with respect to FIGS. 2E, 2F and 2G.

Adapter 220 may include two longitudinal halves 221. Each of longitudinal halves 221 may include a recess 224. Longitudinal halves 221 may be shaped and sized to embrace and lock at least a portion of a pedicle screw head 82 within recesses 224 thereof. Longitudinal halves 221 may be structured to be tightly inserted into a distal portion 212d of housing 210. Each of longitudinal halves 221 may include a concave indent 226 at its proximal end shaped and sized to receive a portion of orthopedic rod 90. Embodiments of adapter 220 are described below with respect to FIGS. 2H, 2I, 2J and 2K.

Internal bolt 230 may include an external thread 234 mating with internal thread 216 of housing 210. Internal bolt 230 may be structured to be screwed into proximal portion 212b of housing 210. Embodiments of internal bolt 230 are described below with respect to FIGS. 2L, 2M, 2N and 2O.

Spacer 240 may be shaped and sized to be inserted into housing 210 between adapter 220 and internal bolt 230. Spacer 240 may be shaped and sized to be pressed by internal bolt 230 against orthopedic rod 90 when internal bolt 230 is being screwed into housing 210. Spacer 240 may include a concave indent 244 on its bottom flat surface shaped and sized to receive a portion of orthopedic rod 90. Embodiments of spacer 240 are described below with respect to FIGS. 2L, 2M, 2N and 2O.

In some embodiments, device 200 has no spacer 240. In embodiments in which device 200 has no spacer 240, orthopedic rod 90 is locked between adapter 220 and internal bolt 230 when internal bolt 230 is tightly screwed into proximal portion 212b of housing 210.

External fastener 250 may be shaped and sized to tightly surround at least a part of the proximal portion of housing 210. In the embodiments shown in FIGS. 2A, 2B, 2C and 2D, external fastener 250 is a nut having a substantially annular body and an internal thread 254 mating with an external thread 217 of housing 210 so that external fastener 250 may be screwed onto the proximal portion of housing 210. Embodiments of external fastener 250 are described below with respect to FIGS. 2P, 2R and 2S.

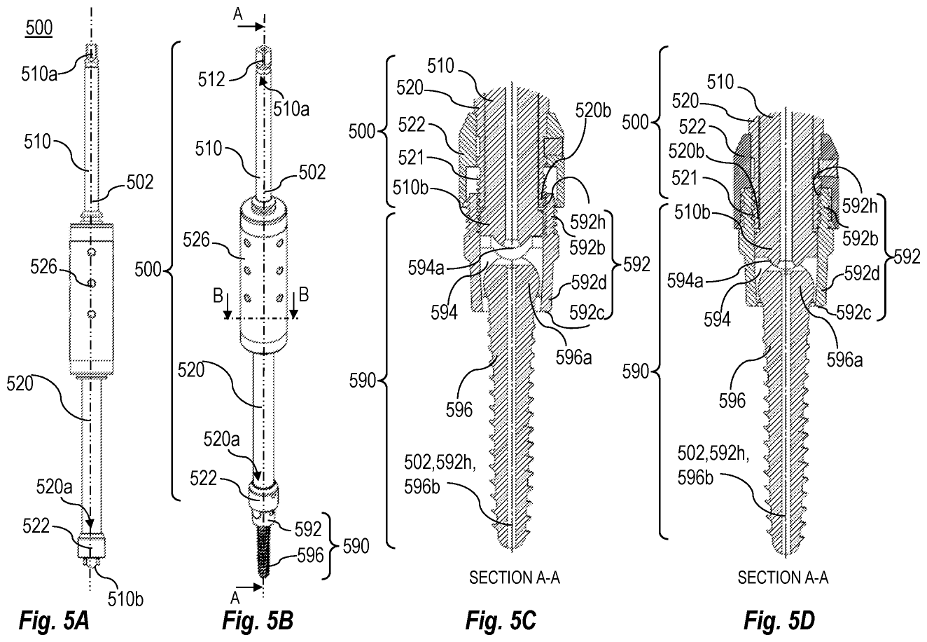

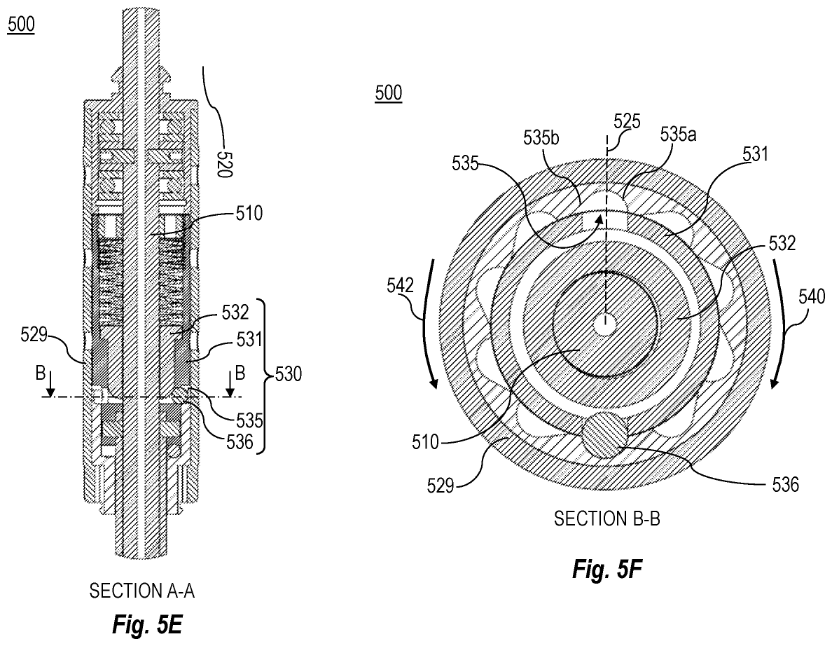

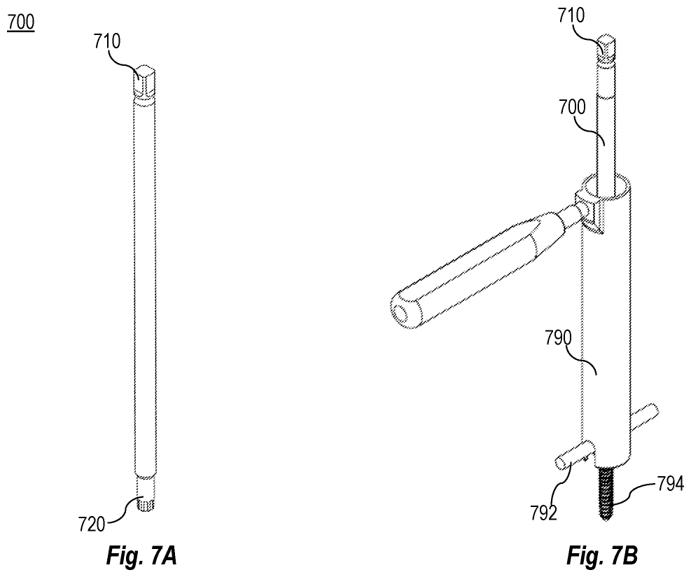

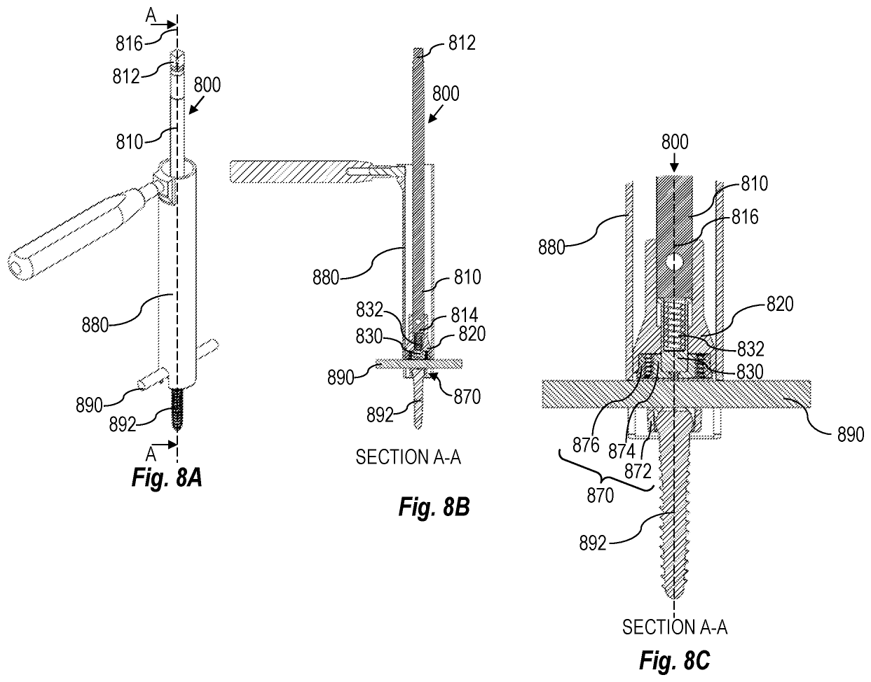

A sequence of functions to be performed to connect orthopedic rod 90 to a pedicle screw 80 using device 200 may include, at a first stage, embracing and locking pedicle screw head 82 between longitudinal halves 221 of adapter 220. At a second stage, tightly inserting longitudinal halves 221 of adapter 220 with pedicle screw head 82 locked therebetween into distal portion 212d of housing 210. The subassembly of pedicle screw 80, housing 210 and adapter 220 may be inserted into a vertebra of a user by, for example, a surgical tool 500 described below with respect to FIGS. 5A, 5B, 5C, 5D, 5E and 5F. At a third stage, inserting orthopedic rod 90 into concave idents 214a, 214b of housing 210 and positioning orthopedic rod 90 into concave indents 226 of longitudinal halves 221 of adapter 220. At a fourth stage, screwing internal bolt 230 into proximal portion 212b of housing 210 so as to receive orthopedic rod 90 within concave indent 244 of spacer 240 (e.g., using a screwing tool 700 described below with respect to FIGS. 7A and 7B). At a fifth stage, screwing external fastener 250 onto proximal portion 212b of housing 210 (e.g., using a screwing tool 800 described below with respect to FIGS. 8A, 8B and 8C). At a sixth stage, simultaneously tightening internal bolt 230 and external fastener 250 to housing 210 (e.g., using a screwing tool 900 described hereinbelow).

Reference is now made to FIGS. 2E, 2F and 2G, which are schematic illustrations of a housing 210 of a device 200 for connecting an orthopedic rod to a pedicle screw, according to some embodiments of the invention.

FIG. 2E shows a schematic perspective view of housing 210. FIG. 2F shows a schematic partial section view of housing 210. FIG. 2G shows a schematic section view of housing 210.

Housing 210 may be made of the composite material. Housing 210 may have a substantially annular body and may have a proximal end 212a, a proximal portion 212b, a distal end 212c and a distal portion 212d.

Housing 210 may include a first concave indent 214a and a second concave indent 214b at its proximal end 212a. First concave indent 214a and second concave indent 214b may be in a longitudinal direction 212e along housing 210. First concave indent 214a and second concave indent 214b may be opposite to each other. First concave indent 214a and second concave indent 214b may be shaped and sized to receive an orthopedic rod.

In some embodiments, an internal surface 212da of distal portion 212d of housing 210 tapers in longitudinal direction 212e of housing 210. Internal surface 212da of distal portion 212d may be shaped and sized to tightly receive adapter 220.

Housing 210 may include an internal thread 216. Internal thread 216 may be on an internal surface of proximal portion 212b of housing 210.

In some embodiments, housing 210 includes an external thread 217 (e.g., as shown in FIGS. 2E, 2F and 2G). External thread 217 may be on an external surface of proximal portion 212b of housing 210.

In some embodiments, internal thread 216 is a right-hand thread, and external thread 217 is a left-hand thread (e.g., as shown in FIGS. 2E, 2F and 2G). In some embodiments, internal thread 216 is a left-hand thread, and external thread 217 is a right-hand thread. In some embodiments, both internal thread 216 and external thread 217 are right-hand threads. In some embodiments, both internal thread 216 and external thread 217 are left-hand threads.

In some embodiments, at least one of internal thread 216 and external thread 217 are coated with a coating material. The coating material may, for example, include PEEK or metal. The coating layer may have a thickness of, for example, 2.5-40 μm. The coating material may, for example, reduce a friction coefficient of the threads.

In some embodiments, housing 210 includes a first slot aperture 218a and a second slot aperture 218b. First slot aperture 218a and second slot aperture 218b may be at distal portion 212d of housing 210. First slot aperture 218a and second slot aperture 218b may be opposite to each other. First slot aperture 218a and second slot aperture 218b may be shaped and sized to receive protrusions of adapter 220 when adapter 220 is inserted into distal portion 212d of housing 210.

In some embodiments, first slot aperture 218a and second slot aperture 218b are elongated in longitudinal direction 212e of housing 210. Elongated first and second slot apertures 218a, 218b may enable adapter 220 to be slidably inserted into distal portion 212d of housing 210.

Slot apertures 218a, 218b and protrusions of adapter 220 may ensure proper positioning of adapter 220 within distal portion 212d of housing 210. Slot apertures 218a, 218b and protrusions of adapter 220 may prevent, or substantially prevent, rotation of adapter 220 with respect to housing 210 when adapter 220 is properly positioned within distal portion 212d of housing 210.

In some embodiments, housing 210 includes an internal composite material layer 219a and an external composite material layer 219b. Fibers of the composite material may be arranged in different directions in internal composite material layer 219a and in external composite material layer 219b.

For example, in embodiments shown in FIG. 2F, in internal composite material layer 219a, fibers of the composite material are arranged in a tangential direction (e.g., substantially along a circumference of housing 210), and in external composite material layer 219b, fibers of the composite material are arranged in an axial direction (e.g., substantially along longitudinal direction 212e of housing 210).

In another example, in internal composite material layer 219a, fibers of the composite material may be arranged in the axial direction, and in external composite material layer 219b, fibers of the composite material may be arranged in the tangential direction. Tangentially arranged fibers of the composite material may withstand relatively high radial forces being applied on housing 210.

In another example, fibers of the composite material may be helically arranged along the length of housing 210. For example, housing 210 may have a single layer of the composite material in which fibers of the composite material are helically arranged along the length of housing 210.

Reference is now made to FIGS. 2H, which is a schematic illustration of an adapter 220 of a device 200 for connecting an orthopedic rod to a pedicle screw, according to some embodiments of the invention.

Reference is also made to FIGS. 2I, 2J and 2K, which are schematic illustrations of a longitudinal half 221 of an adapter 220 of a device 200 for connecting an orthopedic rod to a pedicle screw, according to some embodiments of the invention.

FIG. 2H shows a schematic perspective view of adapter 220. FIG. 2I shows a schematic perspective view of a longitudinal half 221 of adapter 220. FIGS. 2J and 2K show different schematic side views of longitudinal half 221 of adapter 220.

Adapter 220 may include two longitudinal halves 221. Each of longitudinal halves 221 may be made of the composite material. Each of longitudinal halves 221 may have a proximal end 222a, a distal end 222c, a flat longitudinal surface 222e and a curved longitudinal surface 222f.

Longitudinal halves 221 may be shaped and sized to be tightly inserted into an interior 212f of housing 210 when longitudinal halves 221 are positioned in a proximity to each other and are aligned with respect to their flat longitudinal surfaces 222e thereof. For example, longitudinal halves 221 may be shaped and sized to be tightly inserted into distal portion 212d of housing 210 when longitudinal halves 221 are positioned in a proximity to each other and to be aligned with respect to their flat longitudinal surfaces 222e. In some embodiments, curved longitudinal surfaces 222f of each of longitudinal halves 221 tapers in a longitudinal direction 222g of the respective halve. The tapering of curved longitudinal surfaces 222f of longitudinal halves 221 may mate with the tapering of internal surface 212da of distal portion 212d of housing 210.

Each of longitudinal halves 221 may include a recess 224 on flat longitudinal surface 222e thereof. Recess 224 of each of longitudinal halves 221 may extend from distal end 222c towards proximal end 222a in longitudinal direction 222g along a portion of a length of the respective longitudinal halve.

Longitudinal recesses 224 of longitudinal halves 221 may be shaped and sized so as to receive and lock at least a portion of a head of a pedicle screw when longitudinal halves 221 are positioned in a proximity to each other and aligned with respect to their flat longitudinal surfaces 222e.

In some embodiments, proximal end 222a of each of longitudinal halves 221 includes a concave indent 226 in direction 222h of the respective halve. Concave indent 226 of each of longitudinal halves 221 may extend along the entire transverse dimension 222h of proximal end 222a of the respective longitudinal halve. Concave indent 226 of each of longitudinal halves 221 may be shaped and sized to receive a portion of the orthopedic rod.

In some embodiments, each of longitudinal halves 221 includes at least one half protrusion at curved longitudinal surface 222f thereof. The half protrusion(s) of each of longitudinal halves 221 may be shaped, sized and positioned to be slidably and tightly insertable into one of slot apertures 218a, 218b of housing 210.

In embodiments shown in FIGS. 2H, 2I, 2J and 2K, each of longitudinal halves 221 includes a first half protrusion 228a and a second half protrusion 228b. First half protrusion 228a and second half protrusion 228b of each of longitudinal halves 221 may be opposite to each other. First half protrusion 228a and second half protrusion 228b of each of longitudinal halves 221 may be adjacent to proximal end 222a of the respective longitudinal half.

When longitudinal halves 221 are positioned in a proximity to each other and aligned with respect to their flat longitudinal faces 222e, first half protrusions 228a of longitudinal halves 221 form a first adapter protrusion and second half protrusions 228b of longitudinal halves 221 form a second adapter protrusion. First half protrusion 228a and second half protrusion 228b of each of longitudinal halves 221 may be shaped, sized and positioned so that the first adapter protrusion and the second adapter protrusion are slidably and tightly insertable into first slot aperture 218a and second slot aperture 218b of housing 210, respectively. When protrusions 228a and 228b of each of longitudinal halves 221 enter into slot apertures 218a and 218b, respectively, of housing 210, protrusions 228a, 228b may hold a sub-assembly of pedicle screw 80, housing 210 and two longitudinal halves 221 together.

In embodiments shown in FIGS. 2H, 2I, 2J and 2K, adapter 220 includes two longitudinal halves 221. It is noted that, in some embodiments, adapted 220 may include more than two longitudinal members structured to embrace and lock the pedicle screw head when tightly inserted into distal portion 212d of housing 212. For example, adapter 220 may include four longitudinal members structured to embrace and lock the pedicle screw head when tightly inserted into distal portion 212d of housing 212.

Reference is now made to FIGS. 2L, 2M, 2N and 2O, which are schematic illustrations of an internal bolt 230 and a spacer 240 of a device 200 for connecting an orthopedic rod to a pedicle screw, according to some embodiments of the invention.

FIGS. 2L, 2M and 2N show different schematic perspective views of internal bolt 230 and spacer 240. FIG. 2O shows a schematic section A-A view of internal bolt 230 and spacer 240.

Internal bolt 230 may be made of the composite material. Internal bolt 230 may have a substantially annular body and may have a proximal end 232a and a distal end 232b. Internal bolt 230 may include an external thread 234 on an external lateral surface of internal bolt 230. Thread 234 may mate with internal thread 216 of housing 210. A profile of internal thread 216 of housing 210 may be shaped so as not cause (or substantially not cause) outward radial forces during screwing of internal bolt 230. A profile of internal thread 216 of housing 210 may be shaped to cause internal radial forces towards axis 232c during screwing of internal bolt 230. In some embodiments, thread 234 is coated with a coating material. The coating material may, for example, include PEEK or metal. The coating layer may have a thickness of, for example, 2.5-40 μm. The coating material may, for example, reduce a friction coefficient of thread 234.

Internal bolt 230 may include a tool connector 236 to connect a screwing tool to internal bolt 230. In some embodiments, tool connector 236 is at proximal end 232a of internal bolt 230. Connector 236, for example, have a shape of multi longitudinal slots or torx or a polynomic shape like hex.

Spacer 240 may be made of the composite material. Spacer 240 may be substantially flat and may have an upper flat surface 242a and a bottom flat surface 242b. Spacer 240 may include a concave indent 244 on bottom flat surface 242b thereof. Concave indent 244 may extend along the entire longitudinal dimension 242c of spacer 240. Concave indent 244 may be shaped and sized to receive a portion of the orthopedic rod.

In some embodiments, spacer 240 includes a central circular portion 242d. Central circular portion 242d of spacer 240 may be shaped and sized to be inserted into an interior of housing 210.

In some embodiments, spacer 240 includes two radial protrusion 242e radially protruding from central circular portion 242d. In some embodiments, radial protrusions 242e are opposite to each other. Radial protrusions 242e of spacer 240 may be shaped and sized to be inserted into first concave ident 214a and second concave indent 214b of housing 210.

In some embodiments, each of radial protrusions 242e includes two lateral portions 242ea laterally protruding from opposite lateral sides of a distal end of the respective radial protrusion.

In some embodiments, upper flat surface 242a of spacer 240 is coated with a coating material at least in central circular portion 242d of spacer 240. In some embodiments, upper flat surface 242a of spacer 240 is coated with a coating material at least at distal ends of radial protrusions 242e of spacer 240. The coating material may, for example, include PEEK or metal (e.g., Titanium or Titanium alloy). The coating layer may have a thickness of, for example, 2.5-40 μm. The coating material may, for example, reduce a friction coefficient of between spacer 240 and internal bolt 230 and between spacer 240 and external fastener 250.

In some embodiments, spacer 240 is rotatably connected to internal bolt 230. In some embodiments, spacer 240 is connected at its center point 242da to distal end 232b of internal bolt 230 and rotatable with respect to internal bolt 230 about a central longitudinal axis 232c of internal bolt 230. For example, spacer 240 may be rotatably connected to internal bolt 230 using a hinge 238. Hinge 238 may be made of, for example, PEEK.

Reference is now made to FIGS. 2P, 2Q and 2R, which are schematic illustrations of an external fastener 250, according to some embodiments of the invention.

External fastener 250 may be made of the composite material. External fastener 250 may have a substantially annular body and may have a proximal end 252a and a distal end 252b.

In some embodiments, external fastener 250 includes an internal thread 254 on an internal surface of external fastener 250. Internal thread 254 of external fastener 250 may mate with external thread 217 of housing 210. A profile of internal thread 254 may be shaped so as to not cause (or substantially not cause) internal radial forces towards a symmetric axis of external fastener 250 during the screwing thereof.

In some embodiments, internal thread 254 of external fastener 250 is coated with a coating material. The coating material may, for example, include PEEK or metal. The coating layer may have a thickness of, for example, 2.5-40 μm. The coating material may, for example, reduce a friction coefficient of between internal thread 254 of external fastener 250 and external thread 217 of housing 210. The direction of external thread 217 may be left-hand or right-hand.

External fastener 250 may include a tool connector 256 to connect fastener 250 to a screwing tool. For example, tool connector 256 may be at proximal end 252a of external fastener 250. For example, tool connector 256 may be along a portion of external lateral surface of fastener 250.

In some embodiments, distal end 252b of external fastener 250 is shaped to contact spacer 240. In these embodiments, distal end 252b of external fastener 250 may be coated with a coating material. The coating material may, for example, include PEEK or metal. The coating layer may have a thickness of, for example, 2.5-40 μm.

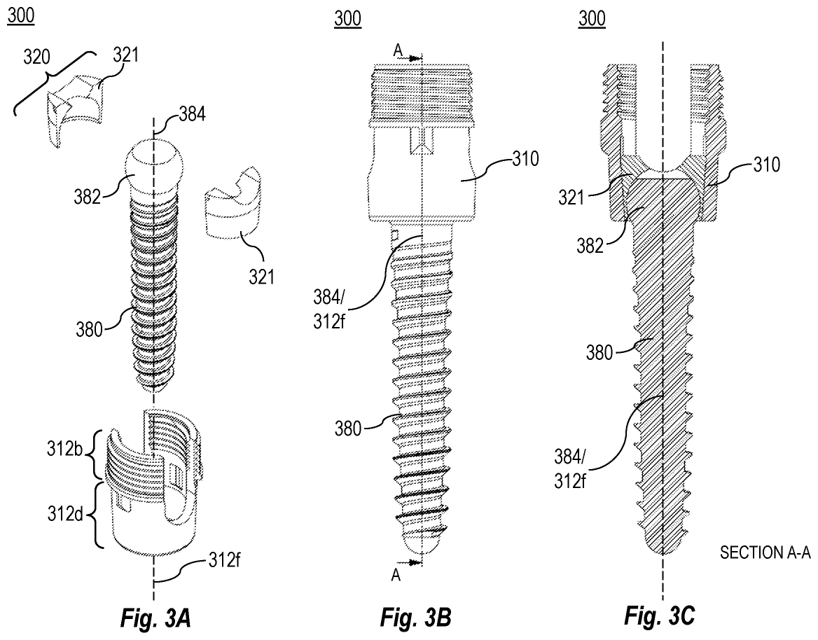

Reference is now made to FIGS. 3A, 3B and 3C, which are schematic illustrations of a pedicle screw sub-assembly 300 including a housing 310, an adapter 320 and a pedicle screw 380, according to some embodiments of the invention.

FIG. 3A shows an exploded schematic perspective view of pedicle screw sub-assembly 300. FIG. 3B shows a schematic side view of assembled pedicle screw sub-assembly 300. FIG. 3C shows a schematic section A-A view of assembled pedicle screw sub-assembly 300.

According to some embodiments of the invention, pedicle screw sub-assembly 300 includes a housing 310, an adapter 320 and a pedicle screw 380. Housing 310, adapter 320 and pedicle screw 380 may be made of the composite material.

Housing 310 may be similar to housing 210 of device 200 described above with respect to FIGS. 2E, 2F and 2G. Housing 310 may have a substantially annular body, a proximal portion 312b, a distal portion 312d and a central longitudinal axis 312f.

Adapter 320 may be similar to adapter 220 of device 200 described above with respect to FIGS. 2H, 2I, 2J and 2K. Adapter 320 may include two longitudinal halves 321. Longitudinal halves 321 may be positioned within distal portion 312d of housing 310 and may embrace and lock at least a portion of a head 382 of pedicle screw 380 such that a central longitudinal axis 384 of pedicle screw 380 coincides with a central longitudinal axis 312f of housing 310. Longitudinal halves 321 of adapter 320 are prepressed in a longitudinal direction of housing 310 so as to maintain the coincidence of central longitudinal axis 384 of pedicle screw 380 with central longitudinal axis 312f of housing 310.

Pedicle screw sub-assembly 300 may be assembled at, for example, manufacturer site. Pedicle screw sub-assembly 300 may be implanted into a vertebra of a subject using a surgical tool (e.g., surgical tool 500 described below with respect to FIGS. 5A, 5B, 5C, 5D, 5E and 5F).

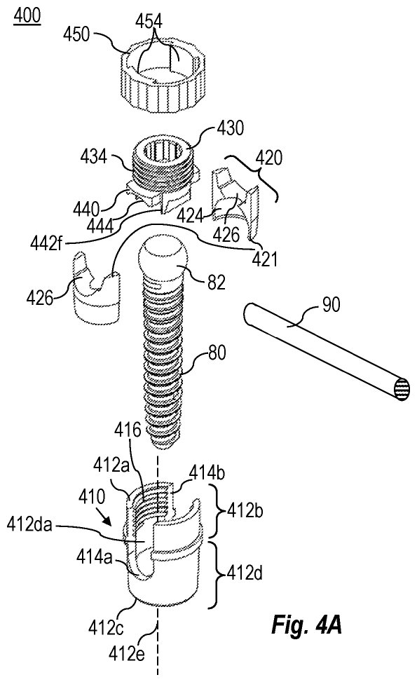

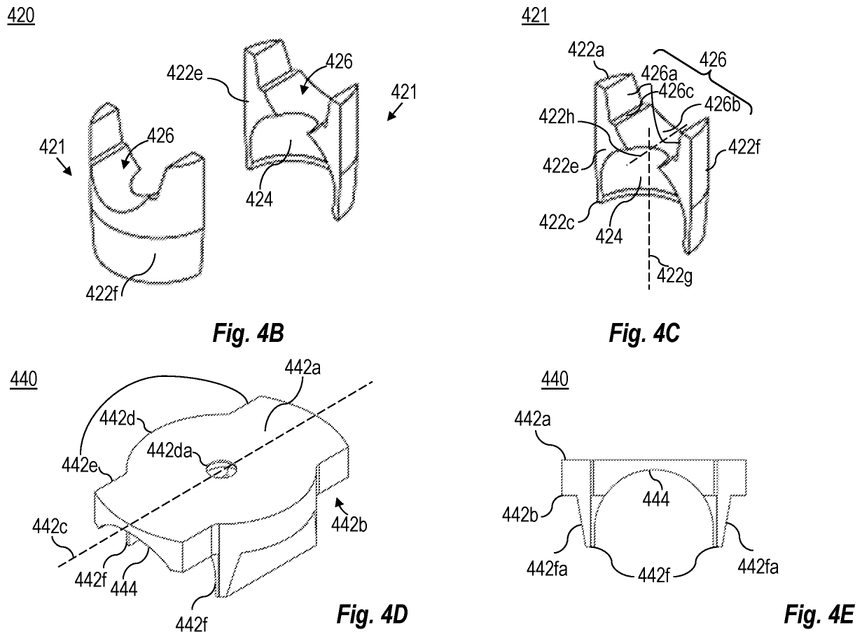

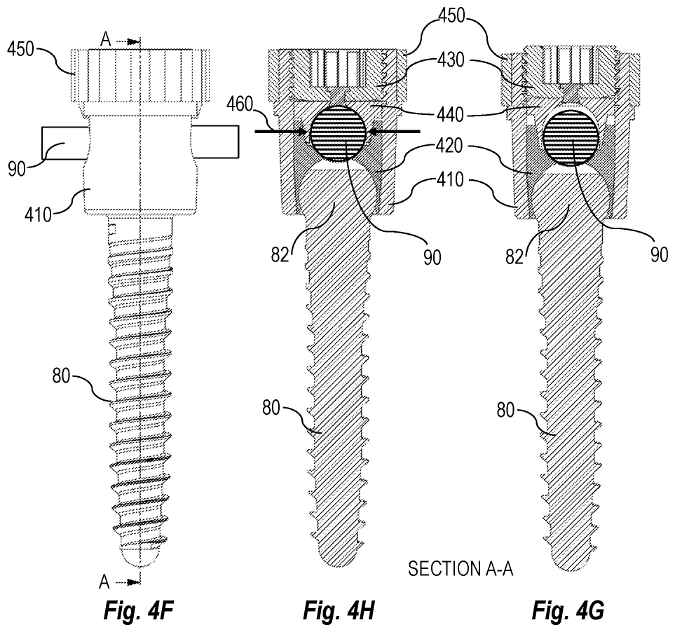

Reference is now made to FIG. 4A, which is a schematic illustration of a device 400 for connecting an orthopedic rod 90 to a pedicle screw 80, according to some embodiments of the invention.

According to some embodiments of the invention, device 400 for connecting an orthopedic rod to a pedicle screw includes a housing 410, an adapter 420, an internal bolt 430, a spacer 440 and an external fastener 450.

Housing 410, adapter 420, internal bolt 430, spacer 440 and external fastener 450 according to some embodiments of the present invention are made of the composite material. In some embodiments, the composite material includes polyether ether ketone (PEEK). In some embodiments, the composite material includes carbon fibers. In some embodiments, the composite material includes PEEK and carbon fibers. For example, the composite material may include 40% PEEK and 60% carbon fibers.

Housing 410 may have a substantially annular body. Housing 410 may include two opposite concave indents 414a, 414b at its proximal end 412a in a longitudinal direction thereof. Concave indents 414a, 414b may be shaped and sized to receive an orthopedic rod.

Housing 410 may include an internal thread 416 on an internal surface of a proximal portion 412b of housing 410. In some embodiments, internal thread 416 is coated with a coating material. The coating material may, for example, include PEEK or metal. The coating layer may have a thickness of, for example, 2.5-40 μm. The coating material may, for example, reduce a friction coefficient of threads.

In some embodiments, an internal surface 412da of distal portion 412d of housing 410 tapers in a longitudinal direction 412e of housing 410. Internal surface 412da of distal portion 412d may be shaped and sized to tightly receive adapter 420.

In some embodiments, housing 410 includes an internal composite material layer and an external composite material layer, wherein fibers of the composite material are arranged in different directions in the internal composite material layer and in the external composite material layer (e.g., as described above with respect to FIGS. 2E, 2F and 2G). In some embodiments, in one layer of the internal composite material layer and the external composite material layer, fibers of the composite material are arranged in a tangential direction, and in another layer of the internal composite material layer and the external composite material layer, fibers of the composite material are arranged in an axial direction (e.g., as described above with respect to FIGS. 2E, 2F and 2G). In some embodiments, fibers of the composite material are helically arranged along the length of the housing (e.g., as described above with respect to FIGS. 2E, 2F and 2G).

Housing 410 may be similar to housing 210 described above with respect to FIGS. 2E, 2F and 2G, with an exception that housing 410 has no external thread on an external lateral surface of proximal portion 412b thereof.

Adapter 420 may include two longitudinal halves 421. Each of longitudinal halves 421 may include a recess 424. Longitudinal halves 421 may be shaped and sized to embrace and lock at least a portion of a pedicle screw head 82 within recesses 424 thereof. Longitudinal halves 421 may be shaped and sized to be tightly inserted into a distal portion 412d of housing 410. Each of longitudinal halves 421 may include a partially concave indent 426 at its proximal end shaped and sized to receive a portion of orthopedic rod 90 and a portion of spacer 440. Embodiments of adapter 420 are described below with respect to FIGS. 4B and 4C.

Internal bolt 430 may include an external thread 434 mating with internal thread 416 of housing 410. Internal bolt 430 may be structured to be screwed into the proximal portion of housing 410. Internal bolt may be similar to internal bolt 230 described above with respect to FIGS. 2L, 2M, 2N and 2O.

Spacer 440 may be shaped and sized to be inserted into housing 410 between adapter 420 and internal bolt 430. Spacer 440 may be shaped and sized to be pressed by internal bolt 430 against adapter 420 when internal bolt 430 is being screwed into housing 210. Spacer 240 may include a concave indent 444 on its bottom surface structured to receive a portion of orthopedic rod 90. Spacer 440 may include spacer protrusions 442f shaped and sized to be tightly inserted into a portion of partially concave indents 426 of longitudinal halves 421 of adapter 420. Embodiments of spacer 440 are described below with respect to FIGS. 4D and 4E.

External fastener 450 may be structured to tightly surround at least a part of the proximal portion of housing 410. In embodiments shown in FIGS. 4A, 4F, 4G and 4H, external fastener 450 is a substantially annular body optionally having two opposite protrusions 454 protruding inwardly from an inner lateral surface of external fastener 450. Protrusions 454 may be shaped and sized to be inserted into concave indents 414a, 414b in housing 410 when external fastener is inserted on proximal portion 412b of housing 410.

A sequence of functions to be performed to connect orthopedic rod 90 to a pedicle screw 80 using device 400 may include, at a first stage, embracing and locking pedicle screw head 82 between longitudinal halves 421 of adapter 420. At a second stage, tightly inserting longitudinal halves 421 of adapter 420 with pedicle screw head 82 locked therebetween into distal portion 412d of housing 410 (e.g., using a surgical tool 500 described below with respect to FIGS. 5A, 5B, 5C, 5D, 5E and 5F). At a third stage, inserting orthopedic rod 90 into concave idents 414a, 414b of housing 410, and positioning the orthopedic rod into concave indents 426 of halves 421 of adapter 420. At a fourth stage, inserting external fastener 450 onto proximal portion 412b of housing 410. At a fifth stage, screwing internal bolt 430 into proximal portion 412b of housing 410 so as to receive orthopedic rod 90 within concave indent 444 of spacer 440 (e.g., using a screwing tool 700 described below with respect to FIGS. 7A and 7B). At a sixth stage, tightening internal bolt 430 to housing 410.

Reference is now made to FIG. 4B, which is a schematic illustration of an adapter 420 of a device 400 for connecting an orthopedic rod to a pedicle screw, according to some embodiments of the invention.

Reference is also made to FIG. 4C, which is a schematic illustration of a longitudinal halve 421 of an adapter 420 of a device 400 for connecting an orthopedic rod to a pedicle screw, according to some embodiments of the invention.

FIG. 4B shows a schematic perspective view of adapter 420. FIG. 4C shows a schematic perspective view of a longitudinal halve 421 of adapter 420.

Adapter 420 may include two longitudinal halves 421. Each of longitudinal halves 421 may be made of the composite material. Each of longitudinal halves 421 may have a proximal end 422a, a distal end 422c a flat longitudinal surface 422e and a curved longitudinal surface 422f.

Longitudinal halves 421 may be shaped and sized to be inserted into an interior of housing 410 when longitudinal halves 421 are positioned in a proximity to each other and aligned with respect to their flat longitudinal surfaces 422e. In some embodiments, curved longitudinal surfaces 422f of each of longitudinal halves 421 tapers in a longitudinal direction 422g of the respective halve. The tapering of curved longitudinal surfaces 422f of longitudinal halves 421 may mate with the tapering of internal surface 412da of distal portion 412d of housing 410.

Each of longitudinal halves 421 may include a recess 424 on flat longitudinal surface 422e thereof. Recess 424 of each of longitudinal halves 421 may extend from distal end 422c towards proximal end 422a in longitudinal direction 422g along a portion of a length of the respective longitudinal halve. Longitudinal recesses 424 of longitudinal halves 421 may be shaped and sized so as to receive and lock at least a portion of a head of a pedicle screw when longitudinal halves 421 are positioned in a proximity to each other and are aligned and aligned with respect to their flat longitudinal surfaces 422e.

In some embodiments, proximal end 422a of each of longitudinal halves 421 includes a concave indent 426 in longitudinal direction 422g of the respective half. Concave indent 426 of each of longitudinal halves 421 may extend along the entire transverse dimension 422h of proximal end 422a of the respective longitudinal half.

Concave indent 426 of each of longitudinal halves 421 may include two opposite sloped surfaces 426a connected at their distal ends to proximal ends of a concave arc 426b. In some embodiments, concave indent 426 includes a flat intermediate surface 426c connecting distal ends of sloped surfaces 426a and proximal ends of concave arc 426b. Flat intermediate surface 426c may be perpendicular to longitudinal direction 422g of the respective longitudinal half. Sloped surfaces 426a, and optionally intermediate surface 426c, of concave indent 426 may be shaped and sized to tightly receive spacer protrusions 442f of spacer 440. Concave arc 426b of concave indent 426 may be shaped and sized to receive at least a portion of the orthopedic rod.

In embodiments shown in FIGS. 4B and 4C, adapter 420 includes two longitudinal halves 421. It is noted that, in some embodiments, adapted 420 may include more than two longitudinal members structured to embrace and lock the pedicle screw head when tightly inserted into distal portion 412d of housing 412. For example, adapter 420 may include four longitudinal members structured to embrace and lock the pedicle screw head when tightly inserted into distal portion 412d of housing 412.

Reference is now made to FIGS. 4D and 4E, which are schematic illustrations of a spacer 440 of a device 400 for connecting an orthopedic rod to a pedicle screw, according to some embodiments of the invention.

FIG. 4D shows a schematic perspective view of spacer 440. FIG. 4E shows a schematic front/rear view of spacer 440.

Spacer 440 may be made of the composite material. Spacer 440 may have an upper flat surface 442a and a bottom flat surface 442b. Spacer 440 may include a concave indent 444 on bottom surface 442b thereof. Concave indent 444 may extend along the entire longitudinal dimension 442c of spacer 440. Concave indent 444 may be shaped and sized to receive a portion of the orthopedic rod.

In some embodiments, spacer 440 includes a central circular portion 442d. Central circular portion 442d of spacer 440 may be shaped and sized to be inserted into an interior of housing 410.

In some embodiments, spacer 440 includes two radial protrusion 442e radially protruding from central circular portion 442d. In some embodiments, radial protrusions 442e are opposite to each other. Radial protrusions 442e of spacer 440 may be shaped and sized to be inserted into first concave ident 414a and second concave indent 414b of housing 410.

In some embodiments, spacer 440 includes two spacer protrusions 442f protruding from central circular portion 442d of bottom flat surface 442b of spacer 440. In some embodiments, spacer protrusions 442f are opposite to each other so that concave indent 444 is between spacer protrusions 442f. Spacer protrusions 442f may be shaped and sized to be inserted into proximal portions 426a of concave indents 426 of longitudinal halves 421 of adapter 420. In some embodiments, each of spacer protrusions 442f has a sloped outer surface 442fa. Sloped outer surfaces 442fa of spacer protrusions 442f may mate with sloped surfaces 426a of concave indents 426 of longitudinal halves 421 of adapter 420.

In some embodiments, upper flat surface 442a of spacer 440 is coated with a coating material at least in central circular portion 442d of spacer 440. In some embodiments, upper flat surface 442a of spacer 440 is coated with a coating material at least at distal ends of radial protrusions 442e of spacer 440. The coating material may, for example, include PEEK or metal. The coating layer may have a thickness of, for example, 2.5-40 μm. The coating material may, for example, reduce a friction coefficient of between elongated plate 442 and internal bolt 430 and distal end 252b of fastener 250.

In some embodiments, spacer 440 is rotatably connected to internal bolt 430. In some embodiments, spacer 440 is connected at its center point 442da to a distal end of internal bolt 230 and rotatable with respect to internal bolt 230 about a central longitudinal axis of internal bolt 430 (e.g., as described above with respect to FIGS. 2L, 2M, 2N and 2O).

Reference is now made to FIGS. 4F, 4G and 4H, which are schematic illustrations of a device 400 for connecting an orthopedic rod 90 to a pedicle screw 80, according to some embodiments of the invention.

FIG. 4F shows side view of assembled device 400 connected to orthopedic rod 90 and pedicle screw 80. FIGS. 4G and 4H show section A-A view of assembled device 400 connected to orthopedic rod 90 and pedicle screw 80.

FIG. 4G shows a schematic illustration of a state in which internal bolt 430 is not tightly screwed into housing 210. FIG. 4H shows a schematic illustration of a state in which internal bolt 430 is tightly screwed into housing 210.

When internal bolt 430 is tightly screwed into housing 410, housing 210, adapter 420 and spacer 440 apply radial forces 460 on opposite sides of orthopedic rod 90 when orthopedic rod 90 is positioned between concave idents 426 and 444 of adapter 420 and spacer 440, respectively, to provide enhanced locking of the orthopedic rod within device 400.

Some embodiments of the present invention provide a pedicle screw sub-assembly including: a composite material pedicle screw; housing 410, adapter 420 having two longitudinal halves 421 positioned within distal portion 412d of housing 410, longitudinal halves 421 embrace and lock a portion of a head of the composite material pedicle screw such that a central longitudinal axis of the composite material pedicle screw coincides with a central longitudinal axis of housing 410; wherein longitudinal halves 421 of adapter 420 are prepressed in longitudinal direction 412e of housing 410 so as to maintain the coincidence of the central longitudinal axis of the composite material pedicle screw with the central longitudinal axis of housing 410.

Reference is now made to FIG. 5A, which is schematic illustrations of a surgical tool 500, according to some embodiments of the invention.

FIG. 5A shows a schematic side view of surgical tool 500.

Reference is also made to FIG. 5B, which is a schematic illustration of a surgical tool 500 connected to a pedicle screw sub-assembly 590, according to some embodiments of the invention.

Reference is also made to FIGS. 5C and 5D, which are schematic illustrations of a section A-A view of a distal end of a surgical tool 500 connected to a pedicle screw sub-assembly 590, according to some embodiments of the invention.

FIG. 5C shows a state at which pedicle screw sub-assembly adapter 594 is not tightly inserted into distal portion 592d of pedicle screw sub-assembly housing 590. FIG. 5D shows a state at which pedicle screw sub-assembly adapter 594 is tightly inserted into distal portion 592d of pedicle screw sub-assembly housing 592.

Surgical tool 500 may be removably connectable to a pedicle screw sub-assembly 590 (e.g., such as pedicle screw sub-assembly 300 described above with respect to FIGS. 3A, 3B and 3D). Surgical tool 500 may further used to implant pedicle screw sub-assembly 590 into a vertebra of a subject.

Pedicle screw sub-assembly 590 may include a housing 592 (e.g., such as housing 210 and 410 described hereinabove), an adapter 594 (e.g., such as adapter 220 and 420 described hereinabove) and a pedicle screw 596. Housing 592, adapter 594 and pedicle screw 596 may be made of the composite material. Adapter 594 may embrace and lock a portion of a pedicle screw head 596a such that a central longitudinal axis 596b of pedicle screw 596 coincides with a central longitudinal axis 592h of housing 592. Adapter 594 may be prepressed in a longitudinal direction of housing 592 so as to maintain the coincidence of central longitudinal axis 596b of pedicle screw 596 with central longitudinal axis 592h of housing 592.

According to some embodiments of the invention, surgical tool 500 includes a tool rod 510, a tool tube 520 and a tool handle 529.

Tool rod 510 may have a proximal end 510a and a distal end 510b. Proximal end 510a of tool rod 510 may be shaped and sized to be removably connectable to a screwing tool handle (e.g., a T-shape screwing tool handle). Distal end 510b of tool rod 510 may have shape and size that mate with shape and size of a proximal end 594a of adapter 594.

Tool tube 520 may have a substantially annular body. Tool tube 520 may accommodate a portion of tool rod 510. For example, tool tube 520 may accommodate a distal portion of tool rod 510, e.g., except distal end 510b of tool rod 510. Tool tube 520 may include an external thread 521 on an external lateral surface of its distal end 520b. External thread 521 of tool tube 520 may mate with internal thread 592h on an internal surface of proximal portion 592b of pedicle screw sub-assembly housing 592.

In some embodiments, tool tube 520 includes an envelope 522. Envelope 522 may surround distal end 520a of tool tube 520 and may be at a radial distance from distal end 520a.

Envelope 522 may be shaped and sized to receive at least a proximal portion 592b of pedicle screw sub-assembly housing 592.

Tool handle 529 may be a substantially annular body. Tool handle 529 may accommodate at least a portion of tool rod 510. Tool handle 529 may be coupled to tool tube 520. Tool handle 529 may be coupled to tool rod 510 so as to enable rotation of tool handle 529 and of tool tube 520 coupled thereto with respect tool rod 510 about a tool central longitudinal axis 502, and to prevent longitudinal motion of tool handle 529 and of tool tube 520 coupled thereto with respect to tool rod 510. In some embodiments, tool handle 529 is coupled to tool rod 510 by a torque limiter.

A sequence of functions to be performed to connect surgical tool 500 to pedicle screw sub-assembly 590 may include, at a first stage, inserting distal end 510b of tool rod 510 into proximal portion 592b of pedicle screw sub-assembly housing 592. At a second stage, rotating tool handle 529, and tool tube 520 coupled thereto, with respect to pedicle screw sub-assembly housing 592 in a screwing direction about tool longitudinal axis 502 to screw distal end 520b of tool tube 520 into proximal portion 592b of pedicle screw sub-assembly housing 592. While distal end 520b of tool tube 520 is being screwed into proximal portion 592a of pedicle screw sub-assembly housing 592, distal end 510b of tool rod 510 pushes pedicle screw sub-assembly adapter 594 (that is locking pedicle screw head 596a) towards a distal end 592c of pedicle screw sub-assembly housing 592 to tightly insert pedicle screw sub-assembly adapter 594 into distal portion 592d of pedicle screw sub-assembly housing 592. Envelope 522 of tool tube 520 may envelope proximal portion 592b of pedicle screw sub-assembly housing 592 as distal end 520b of tool tube 520 is being screwed into pedicle screw sub-assembly housing 592 so as to balance radial forces being applied by tool tube 520 on proximal portion 592a of pedicle screw sub-assembly housing 592.

Upon connection of surgical tool 500 to pedicle screw sub-assembly 590, surgical tool 500 may be used to implant pedicle screw sub-assembly 590 into a vertebra of a subject. Upon implantation of pedicle screw sub-assembly 590 into a vertebra of a subject, surgical tool 500 may be disconnected from pedicle screw sub-assembly 590 by screwing out tool housing 520 from pedicle screw sub-assembly housing 592.

Screwing tool tube 520 into pedicle screw sub-assembly housing 592 may cause damage to one or more components of pedicle screw sub-assembly 590 if a torque being applied on tool tube 520/tool handle 529 exceeds a certain torque threshold. In some embodiments, surgical tool 500 includes a torque limiter 530 structured to decouple tool handle 529 from tool tube 520 so as to terminate screwing of tool tube 520 into pedicle screw sub-assembly housing 592 when the torque being applied on tool handle 529 exceeds a preset torque threshold. During unscrewing of tool tube 520 from pedicle screw sub-assembly housing 592 (e.g., after implantation of pedicle screw sub-assembly 500 is complete), there is no risk of damaging components of pedicle screw sub-assembly 590. Torque limiter 530 may be structured to maintain coupling of tool handle 529 with tool tube 520 during unscrewing of tool tube 520 from pedicle screw sub-assembly housing 592 independent of the torque value being applied on tool tube 520.

Reference is now made to FIGS. 5E and 5F, which are schematic illustrations of a torque limiter 530 of a surgical tool 500, according to some embodiments of the invention.

FIG. 5E shows a schematic section A-A view of surgical tool 500 and torque limiter 530. FIG. 5F shows a schematic section B-B view of surgical tool 500 and torque limiter 530.

In some embodiments, torque limiter 530 mounted within tool handle 529.

In some embodiments, torque limiter 530 includes a spool housing 531 rigidly connected to tool tube 520 and accommodating a spring-loaded spool 532. Spring-loaded spool 532 may be preloaded so as to prevent compression of one or more springs thereof when a longitudinal force applied on spring-loaded spool 532 is below a preset longitudinal force threshold. Spring-loaded spool 532 may be preloaded based on a desired torque threshold.

In some embodiments, torque limiter 530 includes multiple detents 535 between spool housing 531 and tool tube handle 529, and multiple balls 536 each being held within one of detents 535 by spring-loaded spool 532 so as to couple tool tube handle 529 to spool housing 531 and thus to tool tube 520.

In some embodiments, each of detents 535 is asymmetric with respect to a radial direction 525 of tool tube handle 529. Each of detents 535 may include an arc-like surface 535a shaped and sized to accommodate about a half of a diameter of ball 536, and a sloped surface 535b connected to arc-like surface 535a.

When tool tube handle 529 is being rotated with respect to pedicle screw sub-assembly housing 592 in a screwing direction 540, and when a torque being applied on tool handle 529 is below a preset torque threshold, balls 536 are being held within their respective detents 535 by spring-loaded spool 532 so as to couple tool tube handle 529 to spool housing 531 and thus to tool tube 520.

When tool tube handle 529 is being rotated with respect to pedicle screw sub-assembly housing 592 in screwing direction 540, and when the torque being applied on tool handle 529 is above the preset torque threshold, balls 536 may roll over sloped surfaces 535b of respective detents 353 to displace spring-loaded spool 532 and leave their respective detents 535 so as to decouple tool handle 529 from spool housing 531 and thus from tool tube 520 so as to cause tool handle 529 rotate about tool tube 520 and to terminate screwing of tool tube 520 into pedicle screw sub-assembly housing 592.

When tool handle 529 is being rotated with respect to pedicle screw sub-assembly housing 592 in an unscrewing direction 542, arc-like surfaces 535a of detents 535 may prevent from their respective balls 536 to roll thereover so as to prevent from balls 536 to leave their respective detents 535 and to maintain coupling between tool handle 529 and spool housing 531 and tool tube 520 independent of the torque being applied on tool handle 529. This may enable unscrewing tool tube 520 from pedicle screw sub-assembly housing 592 under any required torque conditions.

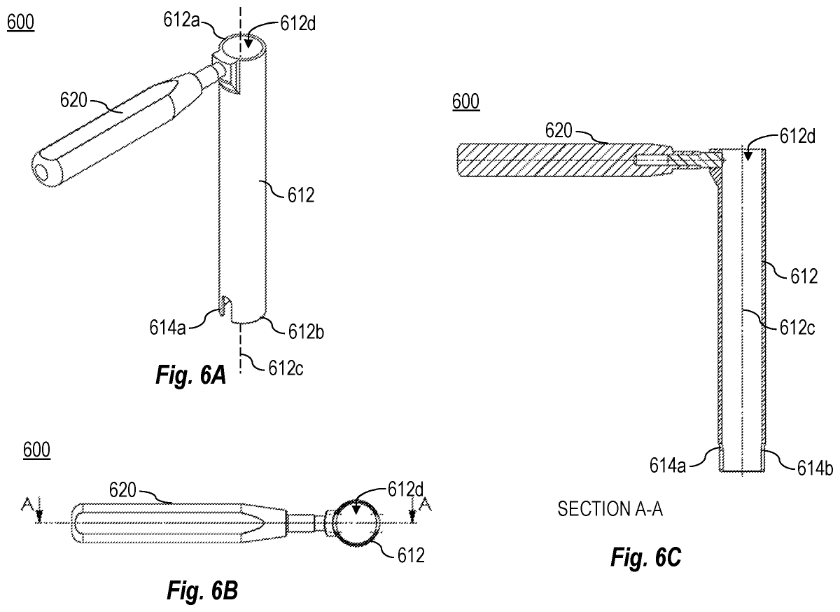

Reference is now made to FIGS. 6A, 6B and 6C, which are schematic illustrations of a device 600 for applying counter torque, according to some embodiments of the invention.

Device 600 may have a substantially annular body 612 and having a proximal end 612a and a distal end 612b. Device 600 may include two opposite concave indents 614a, 614b at distal end 612b of annular body 612 and in a longitudinal direction 612c of annual body 612. Concave idents 614a, 614b may be shaped and sized to receive an orthopedic rod.

Device 600 may include a handle 620 connected to annular body 612. In some embodiments, handle 620 may be perpendicular, or substantially perpendicular, to longitudinal direction 612c of annular body 612. In some embodiments, handle 620 may be at a specified angle to longitudinal direction 612c of annular body 612 (e.g., rather than perpendicular thereto). In some embodiments, handle 620 is connected to an external lateral surface of annular body 612 at proximal end 612a of annular body 612. In some embodiments, annular body 612 includes a handle connector and handle 620 is removably connectable to annular body 612.

Device 600 may be positioned so as to receive an orthopedic rod within concave idents 614a, 614b at its distal end 612b, a screwing tool may be inserted into an interior 612d of device 600 so as to screw components to a pedicle screw sub-assembly implanted into a vertebra of a subject while and a user may hold handle 620 of device 600 that can provide a counter torque to the screwing action being performed by the user.

Reference is now made to FIG. 7A, which is a schematic illustration of a screwing tool 700 for screwing an internal bolt of a device for connecting an orthopedic rod to a pedicle screw, according to some embodiment of the invention.

Reference is also made to FIG. 7B, which is a schematic illustration of a screwing tool 700 for screwing an internal bolt of a device for connecting an orthopedic rod to a pedicle screw, positioned within device 790 for holding a counter torque, wherein device 790 for holding a counter torque is connected to an orthopedic rod 792, according to some embodiments of the invention.

Screwing tool 700 may be a rod having a proximal end 710 and a distal end 720. Proximal end 710 may be shaped and sized to be removably connected to a screwing tool handle (e.g., a T-shape screwing tool handle). Optionally, proximal end 710 may be connectable to a torque limiter and a handle. Distal end 720 may be shaped and sized to be removably connected to a tool connector of an internal bolt of a device for connecting an orthopedic rod to a pedicle screw (e.g., connector 236 of internal bolt 230 described above with respect to FIGS. 2L, 2M, 2N and 2O).

Reference is now made to FIGS. 8A, 8B, 8C and 8D, which are schematic illustrations of an assembly including a screwing tool 800 for screwing a screwable external fastener 876 of a device 870 including a housing 872 and an internal bolt 874 screwed into housing 872, a device 880 for holding a counter torque, an orthopedic rod 890 and a pedicle screw 892, according to some embodiments of the invention.

FIG. 8A shows a schematic perspective view of the assembly. FIG. 8B shows a schematic section A-A view of the assembly. FIG. 8C shows a schematic section A-A view of a distal portion of the assembly.

Screwing tool 80 may be used for screwing a screwable external fastener 876 of a device 870 including a housing 872 and an internal bolt 874 screwed into housing 872. Device 870 may be a device for connecting an orthopedic rod 890 to pedicle screw 892, such as device 200 described above with respect to FIGS. 2A, 2B, 2C and 2D. For example, device 870, housing 872, internal bolt 874 and external fastener 876 may be similar to device 200, housing 210, internal bolt 230 and external fastener 250, respectively, described above with respect to FIGS. 2A, 2B, 2C and 2D, FIGS. 2E, 2F and 2G, FIGS. 2L, 2M, 2N and 2O, and FIGS. 2P, 2R and 2S.

Screwing tool 800 may include a rod 810 having a proximal end 812 and a distal end 814. Proximal end 812 of rod 810 may be shaped and sized to be removably connected to a screwing tool handle or a torque limiter with handle (e.g., a T-shape screwing tool handle).

Screwing tool 800 may include a gripper 820. Gripper 820 may be connected to distal end 814 of rod 810. Gripper 820 may be shaped and sized to grip screwable external fastener 876 of device 870.

Screwing tool 800 may include a spring-loaded pin 830 connected to distal end 814 of rod 810 using a spring 832 along a central longitudinal axis 816 of rod 810. Spring-loaded pin 830 may centralize external fastener 876 of device 870 with respect to internal bolt 874 of device 870 when external fastener 876 is being gripped by gripper 820 and being screwed onto housing 872 of device 870. Centralization of external fastener 876 with respect to internal bolt 874 may cause proper screwing of external fastener 876 onto housing 872 of device 870.

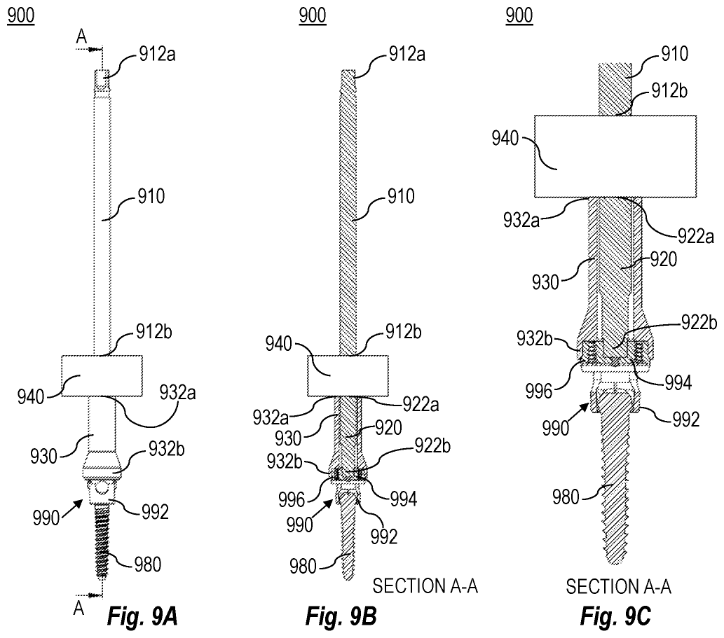

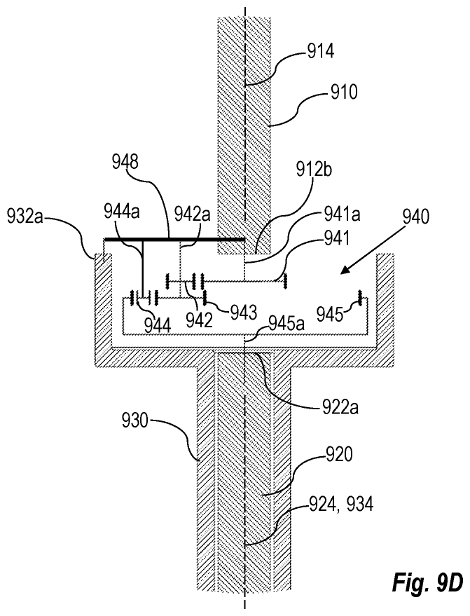

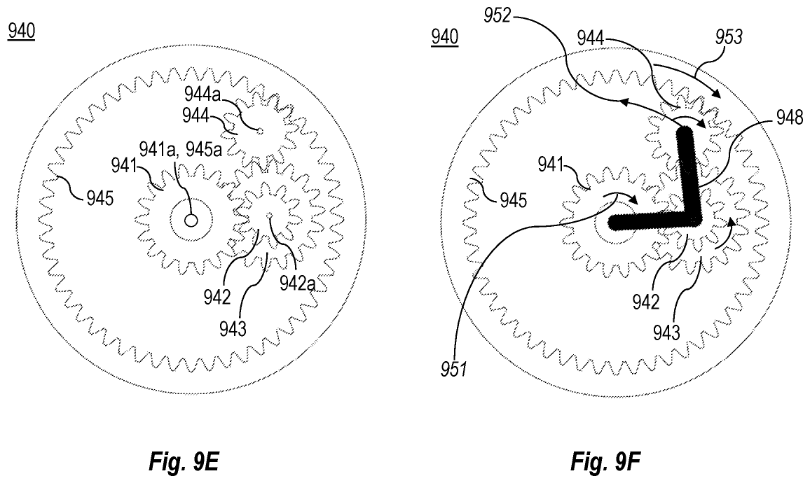

Reference is now made to FIGS. 9A, 9B and 9C, which are schematic illustrations of a screwing tool 900 for simultaneous screwing/tightening of an internal bolt 994 and of an external fastener 996 screwable to a housing 992 of a device 990, according to some embodiments of the invention.

FIG. 9A shows a schematic side view of screwing tool 900. FIGS. 9B and 9C show schematic section A-A views of screwing tool 900.

Device 990 may be, for example, a device for connecting an orthopedic rod to a pedicle screw 980, such as device 200 described above with respect to FIGS. 2A, 2B, 2C and 2D. For example, device 990, housing 992, internal bolt 994 and external fastener 996 may be similar to device 200, housing 210, internal bolt 230 and external fastener 250, respectively, described above with respect to FIGS. 2A, 2B, 2C and 2D, FIGS. 2E, 2F and 2G, FIGS. 2L, 2M, 2N and 2O, and FIGS. 2P, 2R and 2S.

According to some embodiments of the invention, screwing tool 900 includes an input rod 910, an internal bolt rod 920, an external fastener tube 930 and a transmission 940.

Input rod 910 have a proximal end 912a and a distal end 912b. Proximal end 912a of input rod 910 may be shaped and sized to be removably connected to a screwing tool handle (e.g., a T-shape screwing tool handle). Input rod 910 may be coupled at its distal end 912b to transmission 940.

In some embodiments, screwing tool 900 includes a torque limiter. The torque limiter may be coupled to, for example, input rod 910.

Internal bolt rod 920 may have a proximal end 922a and a distal end 922b. Internal bolt rod 920 may be coupled at its proximal end 922a to transmission 940. Distal end 922b of internal bolt rod 920 may be shaped and sized to be removably connected to internal bolt 994 of device 990.

External fastener tube 930 may have a proximal end 932a and a distal end 932b. External fastener 930 may be coupled at its distal end 932a to transmission 940. Distal end 932b of external fastener tube 930 may be shaped and sized to removably grip external fastener 996 of device 900. External fastener tube 930 may accommodate internal bolt rod 920. Internal bolt rod 920 and external fastener tube 930 may be rotatable with respect to each other about their respective central longitudinal axes 924, 934, respectively.

Transmission 940 may be structured to rotate internal bolt rod 920 and external fastener tube 930 about their respective central longitudinal axes 924, 934, respectively, in response to one-directional rotation of input rod 910 about its central longitudinal axis 914.

In embodiments in which internal bolt 994 and external fastener 996 are screwable to housing 992 of device 990 in opposite screwing directions with respect to each other, transmission 940 is structured to rotate internal bolt rod 920 and external fastener tube 930 about their respective central longitudinal axes 924, 934, respectively, in opposite directions with respect to each other in response to one-directional rotation of input rod 910 about its central longitudinal axis 914.

In embodiments in which internal bolt 994 and external fastener 996 are screwable to housing 992 of device 990 in the same screwing direction, transmission 940 is structured to rotate internal bolt rod 920 and external fastener tube 930 about their respective central longitudinal axes 924, 934, respectively, in the same direction in response to one-directional rotation of input rod 910 about its central longitudinal axis 914.

In some embodiments, transmission 940 includes a gear assembly.

A transmission ratio of transmission 940 and directions of rotations of internal bolt rod 920 and of external fastener tube 930 at an output of transmission 940 may be preset based on known screwing directions and known tightening torque values of internal bolt 944 and external fastener 996 of device 960.

For example, in embodiments in which internal bolt 994 and external fastener 996 are screwable to housing 992 of device 990 in opposite screwing directions with respect to each other, transmission 940 may be structured to receive a torque of 3 Newton-Meter (N·m) in a first screwing direction from input rod 910 and to convert it into a torque of 4 N·m being outputted to internal bolt rod 940 in the first screwing direction and into a torque of 7 N·m being outputted to external fastener tube 930 in a second screwing direction that is opposite to the first screwing direction. In this example, an overall tightening torque at an output of transmission 940 (e.g., a torque converted to internal bolt rod 920 and a torque converted to external fastener tube 930) is larger than a torque received from input rod 940. This is balanced by a greater number of rotations being performed by internal rod 910 than number of rotations being performed by at least one of internal bolt rod 920 and external fastener tube 930.

When using screwing tool 900 having transmission 940, a torque transmitted to pedicle screw 980 has the same value and direction as the torque being applied on input rod 910. For example, if a torque of 3 N·m is applied to input rod 940 in a first screwing direction, the torque transmitted to pedicle screw 980 is 3 N·m in the same direction. Accordingly, a counter torque of 3 N·m in opposite to the first screwing direction should be applied to balance the torque being transmitted to pedicle screw 980.

Therefore, the disclosed devices for connecting an orthopedic rod to a pedicle screw and having an internal bolt and an external fastener may have a significant advantage over current typical devices (e.g., utilizing a single fastener), because torques being transmitted to the pedicle screw when using the disclosed devices are significantly lower than those being transmitted when utilizing current typical devices (e.g., 3 N·m when using the disclosed devices as compared to 8-12 N·m when using current typical devices).

Reference is now made to FIG. 9D, which is a schematic illustrations of a section A-A view of a screwing tool 900 for simultaneous screwing/tightening of an internal bolt and an external fastener screwable in opposite screwing directions, including a schematic section A-A view of a transmission 940 of screwing tool 900, according to some embodiments of the invention.

Reference is also made to FIGS. 9E and 9F, which are schematic illustrations of a transmission 940 of a screwing tool 900 for simultaneous screwing/tightening of an internal bolt and an external fastener screwable in opposite screwing directions, according to some embodiments of the invention.

According to some embodiments of the invention, transmission 940 includes a planetary gear assembly.

Transmission 940 may include a sun gear 941. Sun gear 941 may be rigidly connected to input rod 910. For example, sun gear 941 may be rigidly connected to distal end 912b of input rod 910. Sun gear 941 may be rigidly connected to input rod 910 such that a sun gear axis 941a coincides with input rod central longitudinal axis 914. Sun gear 941 and input rod 910 may be rotatable together (e.g., as a single unit) about input rod central longitudinal axis 914 that may coincide with sun gear axis 941a.

Transmission 940 may include a first planet gear 942. First planet gear 942 may be rotatable about a first planet gear axis 942a. First planet gear axis 942a may be parallel, or substantially parallel, to sun gear axis 941a. First planet gear 942 may be meshed with sun gear 941.

Transmission 940 may include a second planet gear 943. Second planet gear 943 may be rigidly connected to first planet gear 942 on first planet gear axis 942a. Second planet gear 943 and first planet 942 may be rotatable together (e.g., a single unit) about first planet gear axis 942a.

Transmission 940 may include a third planet gear 944. Third planet gear 944 may be rotatable about a third planet gear axis 944a. Third planet gear axis 944a may be parallel, or substantially parallel, to sun gear axis 941a. Third planet gear 944 may be meshed with second planet gear 943.

Transmission 940 may include a ring gear 945. Ring gear 945 may be meshed with third planet gear 944. Ring gear 945 may be rigidly connected to internal bolt rod 920. Ring gear 945 may be rotatable about a ring gear axis 945a. Ring gear axis 945a may be parallel, or substantially parallel, to sun gear axis 941a. Ring gear axis 945a may coincide with sun gear axis 941a. Ring gear axis 945a may coincide with internal bolt rod central longitudinal axis 924. Ring gear 945 and internal bolt rod 920 may be rotatable together (e.g., as a single unit) about internal bolt rod central longitudinal axis 924 that may coincide with ring gear axis 945a.

Transmission 940 may include a carrier 948. It is noted that carrier 948 is not shown in FIG. 9E for sake of clarity. Carrier 948 may be a rigid structural element. Carrier 948 may be rotatable about sun gear axis 941a (e.g., that may coincide with input rod central longitudinal axis 914, central internal bolt longitudinal axis 924 and external fastener central longitudinal axis 934).

First and second planet gears 942, 943 may be rotatably coupled to carrier 948 on first planet gear axis 942a. Third planet gear 944 may be rotatably coupled to carrier 948 on third planet gear axis 944a. Carrier 948 may be rigidly connected to external fastener tube 930 so as to rotate external fastener tube 930 about external fastener central longitudinal axis 934 when carrier 948 rotates about sun gear axis 941a (e.g., that may coincide with input rod central longitudinal axis 914, central internal bolt longitudinal axis 924 and external fastener central longitudinal axis 934).

The number of teeth on each of gears 941, 942, 943, 944 and 945 may be preset based on a desired torque transmission ratio of transmission 940.

It is noted that other configurations of transmission 940 are also possible.

FIG. 9F further shows directions of rotations of components of screwing tool 900 and of components of transmission 940. In an example shown in FIG. 9F, internal rod 910 is being rotated in a first screwing direction as indicated by arrow 951. When a resistance of internal bolt rod 920 is greater than a resistance of external fastener tube 930, ring gear 945 and internal bolt 920 rigidly connected thereto may be stationary while carrier 948 and external fastener tube 930 rigidly connected thereto may rotate in a second screwing direction to tighten external fastener 946, as indicated by arrow 952 in FIG. 9F. When a resistance of external fastener tube 930 is greater than a resistance of internal bolt rod 920, carrier 948 and external fastener tube 930 rigidly connected thereto may be stationary while ring gear 945 and internal bolt 920 rigidly connected thereto may rotate in the first screwing direction to tighten internal bolt 944, as indicated by arrow 953 in FIG. 9F. This process may be repeated until internal bolt 994 and external fastener 996 reach their desired tightening torque values.

In embodiments in which internal bolt 994 and external fastener 996 are screwable to housing 992 of device 990 in the same screwing direction, transmission 940 has no third planet gear 944 and ring gear 945 is meshed with second planet gear 943.

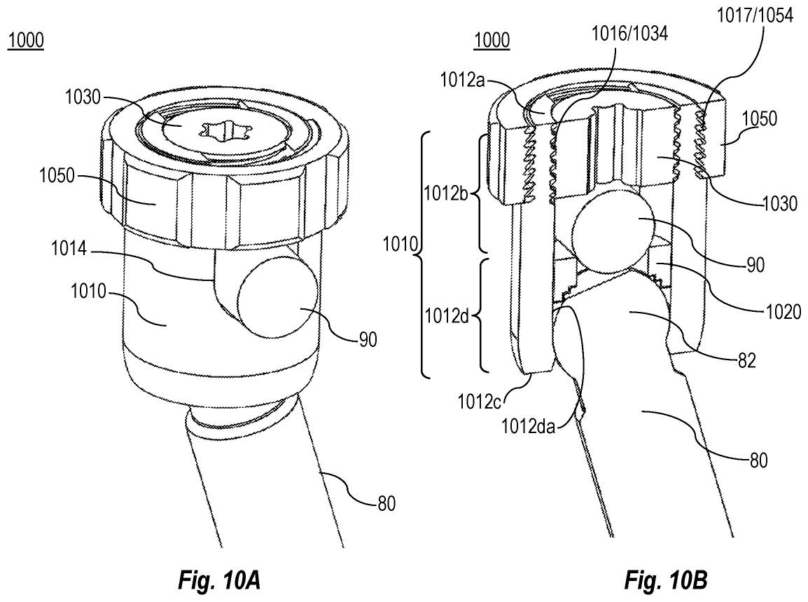

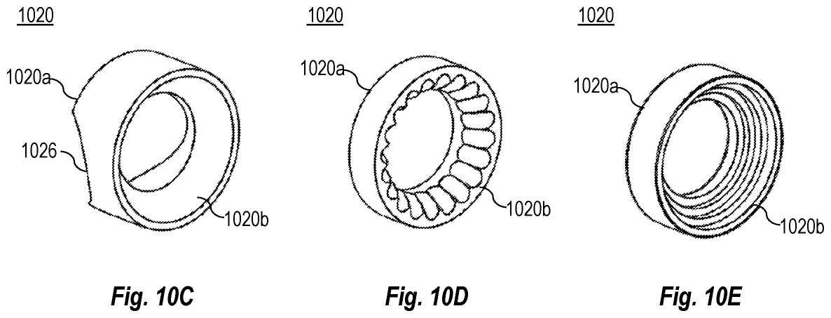

Reference is now made to FIGS. 10A and 10B, which are schematic illustrations of a device 1000 for connecting an orthopedic rod 90 to a pedicle screw 80, according to some embodiments of the invention.

FIG. 10A shows a schematic perspective view of device 1000 connected to orthopedic rod 90 and pedicle screw 80. FIG. 10B shows a schematic section view of device 1000 connected to orthopedic rod 90 and pedicle screw 80

According to some embodiments of the invention, device 1000 for connecting an orthopedic rod 90 to a pedicle screw 80 includes a housing 1010, an adapter 1020, an internal bolt 1030 and an external fastener 1050.

Housing 1010, adapter 1020, internal bolt 1030 and external fastener 1050 according to some embodiments of the present invention are made of the composite material. In some embodiments, the composite material includes polyether ether ketone (PEEK). In some embodiments, the composite material includes carbon fibers. In some embodiments, the composite material includes PEEK and carbon fibers. For example, the composite material may include 40% PEEK and 60% carbon fibers.

Housing 1010 may have a substantially annular body. Housing 1010 may include two opposite concave indents 1014 at its proximal end 1012a in a longitudinal direction thereof. Concave indents 1014 may be shaped and sized to receive an orthopedic rod.

An internal surface 1012da of distal portion 1012d of housing 1010 may be shaped and sized to receive and accommodate pedicle screw head 82. Internal surface 1012da of distal portion 1012d of housing 1010 may be shaped and sized to receive and lock pedicle screw head 82 when pedicle screw head 82 is pressed by adapter 1020.

Housing 1010 may include an internal thread 1016 on an internal surface of a proximal portion 1012b of housing 1010. In some embodiments, housing 1010 includes an external thread 1017 on an external surface of a proximal portion 1012b of housing 1010.

In some embodiments, housing 1010 includes an internal composite material layer and an external composite material layer, wherein fibers of the composite material are arranged in different directions in the internal composite material layer and in the external composite material layer (e.g., as described above with respect to FIGS. 2E, 2F and 2G). In some embodiments, in one layer of the internal composite material layer and the external composite material layer fibers of the composite material are arranged in a tangential direction, and in another layer of the internal composite material layer and the external composite material layer fibers of the composite material are arranged in an axial direction (e.g., as described above with respect to FIGS. 2E, 2F and 2G). In some embodiments, fibers of the composite material are helically arranged along the length of the housing (e.g., as described above with respect to FIGS. 2E, 2F and 2G).

Housing 1010 may be similar to housing 210 described above with respect to FIGS. 2E, 2F and 2G, with an exception that internal surface 1012da of distal portion 1012d of housing 1010 is shaped and sized to receive and pedicle screw head 82 instead of adapter 220 as housing 210.

Adapter 1020 may be shaped and sized to receive a portion of pedicle screw head 82. Adapter 1020 may be shaped and sized to lock pedicle screw head 82 within distal portion 1012d 1010d of housing 1010 when adapted 1020 is being pressed by orthopedic rod 90 and internal bolt 1030. Embodiments of adapter 1020 are described below with respect to FIGS. 10C, 10D, 10E.

Internal bolt 1030 may include an external thread 1034 mating with internal thread 1016 of housing 1010. Internal bolt 1030 may be structured to be screwed into the proximal portion 1012b of housing 1010. When screwed into proximal portion 1012b of housing 1010, internal bolt 1030 may press and lock orthopedic rod 90 between adapter 1020 and internal bolt 1030. Internal bolt may be similar to internal bolt 1030 described above with respect to FIGS. 2L, 2M, 2N and 2O.

External fastener 1050 may be shaped and sized to tightly surround at least a part of the proximal portion of housing 1010. External fastener 1050 may be a nut having a substantially annular body and an internal thread 1054 mating with an external thread 1017 of housing 1010 so as external fastener 1050 may be screwed onto proximal portion 1012b of housing 1010. External fastener 1050 may be similar to external fastener 250 described above with respect to FIGS. 2P, 2R and 2S.

Reference is now made to FIGS. 10C, 10D and 10E, which are schematic illustrations of different configurations of an adapter 1020 of a device 1000 for connecting an orthopedic rod to a pedicle screw, according to some embodiments of the invention.

Adapter 1020 may be a substantially annular body. Adapter 1020 may be shaped and sized to be tightly inserted into housing 1010. Adapter 1020 may lock pedicle screw head 82 within distal portion 1012d of housing 1010 when adapted 1020 is being pressed by orthopedic rod 90 and internal bolt 1030.

Adapter 1020 may be shaped and sized to receive a portion of pedicle screw head 82. In some embodiments, shape and sized of distal end 1020b of adapter 1020 mates with shape and size of a proximal portion of pedicle screw head 82 so as to receive pedicle screw head 82 with a good fitting. FIGS. 10C, 10D and 10E show examples of different shapes of distal ends 1020b of adapter 1020 according to some embodiments of the invention.

In some embodiments, adapter 1020 includes a concave indent 1026 at its proximal end 1020a (e.g., as shown in FIG. 10C). Concave indent 1026 may be shaped and sized to receive orthopedic rod 90.

Reference is now made to FIGS. 11A and 11B, which are schematic illustrations of a pedicle screw implant assembly 1100, according to some embodiments of the invention.

Illustrations 1100aa and 1100ba in FIGS. 11A and 11B, respectively, show side views of pedicle screw implant assembly 1100. Illustrations 1100ab and 1100bb in FIGS. 11A and 111B, respectively, show cross-section views of pedicle screw implant assembly 1100.

Pedicle screw implant assembly 1100 may include an orthopedic rod 1110, a pedicle screw 1120 and a device 1130 for connecting orthopedic rod 1110 to pedicle screw 1120.

In some embodiments, orthopedic rod 1110 is straight (e.g., as shown in FIG. 11A). In some embodiments, orthopedic rod 1110 is bent, curved or otherwise not straight (e.g., as shown in FIG. 11B).

Orthopedic rod 1110, pedicle screw 1120 and device 1130 according to some embodiments of the present invention are made of a composite material. In some embodiments, the composite material includes polyether ether ketone (PEEK). In some embodiments, the composite material includes carbon fibers. In some embodiments, the composite material includes PEEK and carbon fibers. For example, the composite material may include 40% PEEK and 60% carbon fibers.

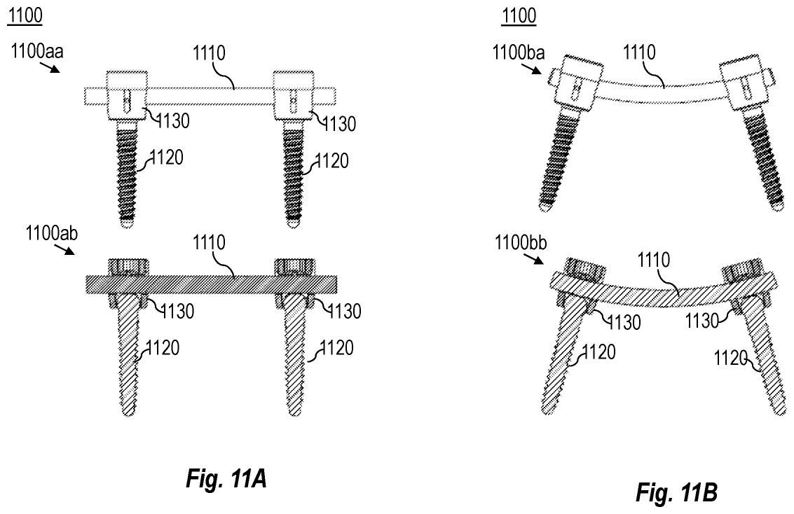

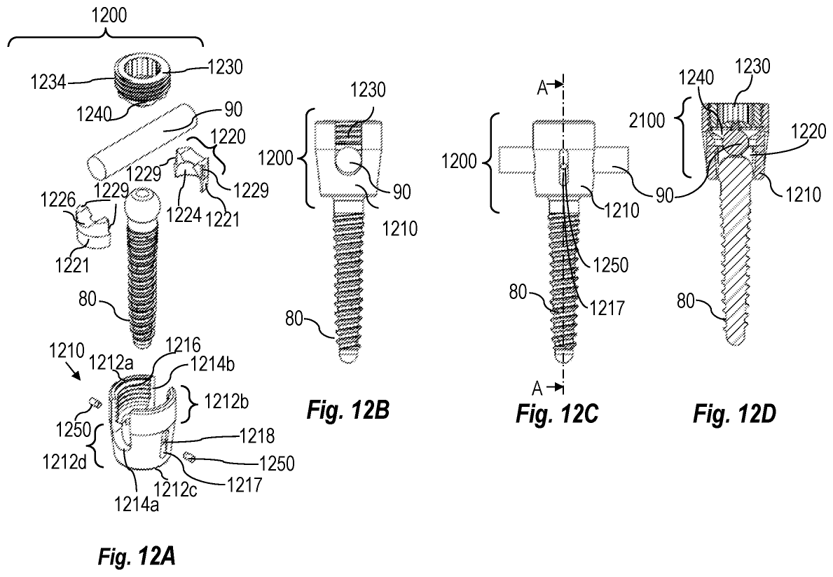

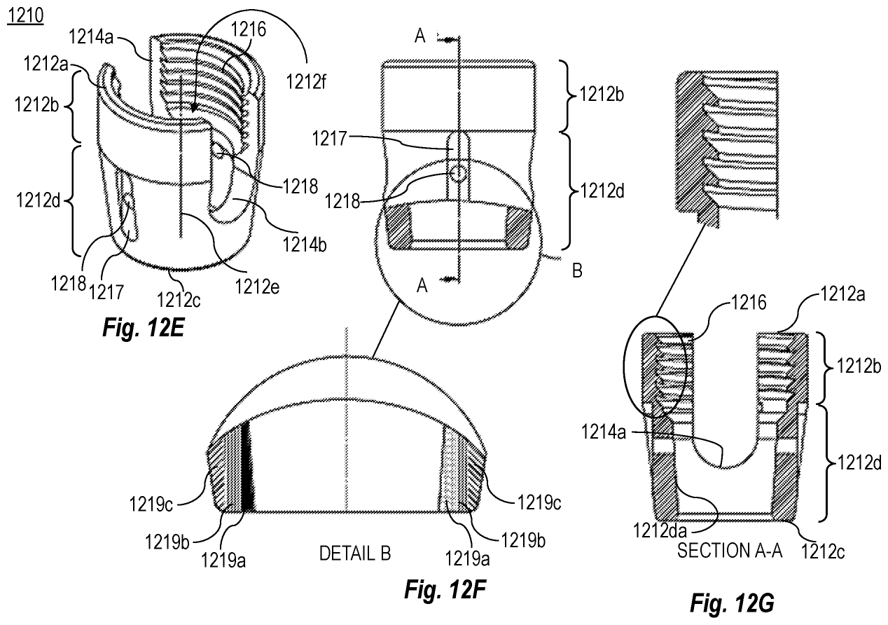

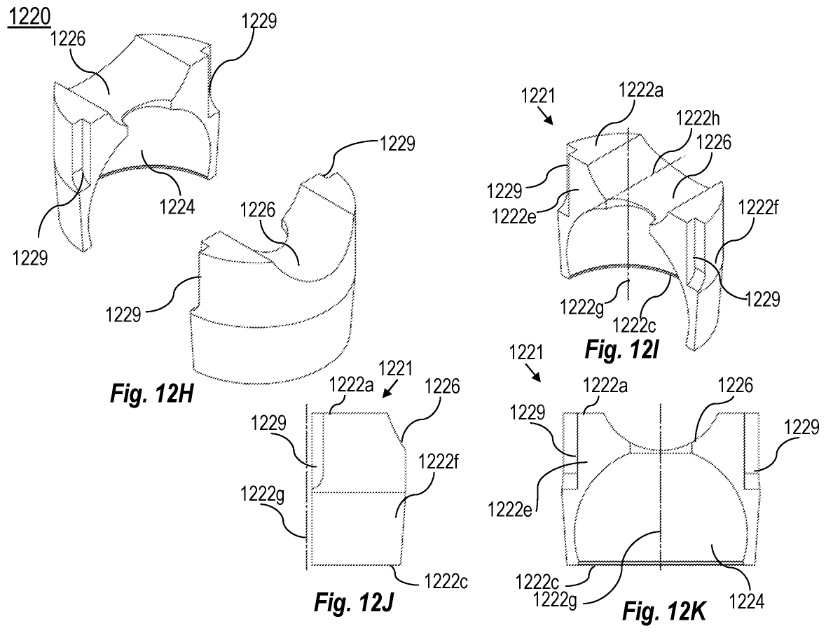

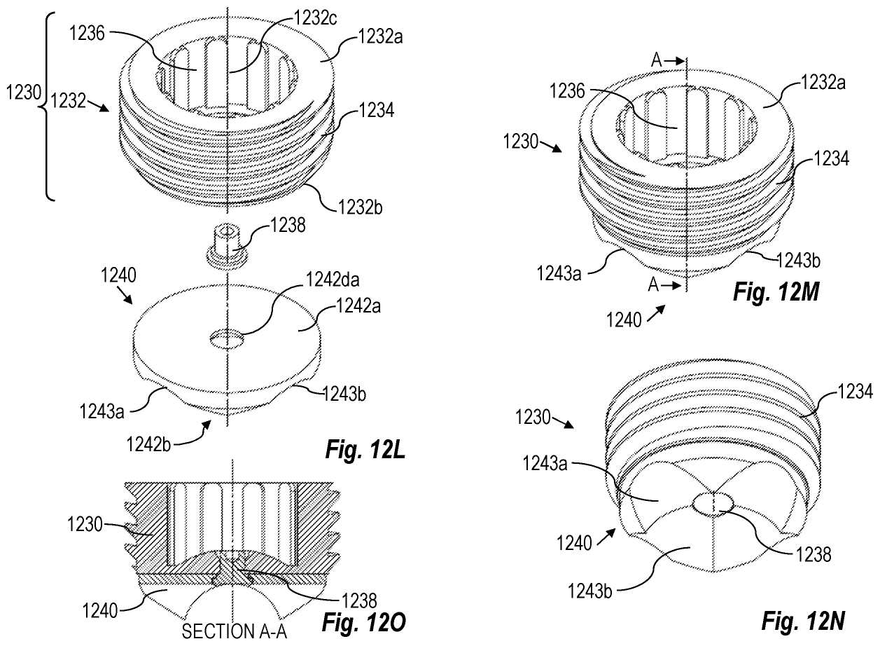

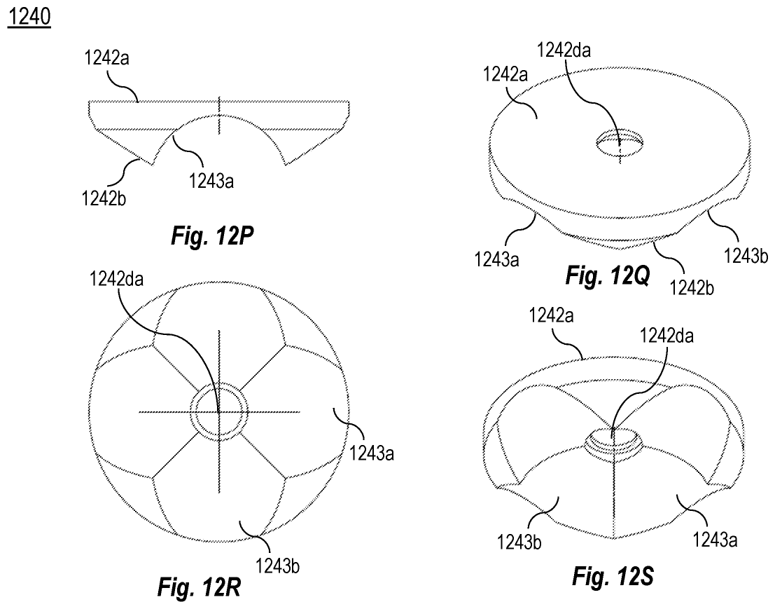

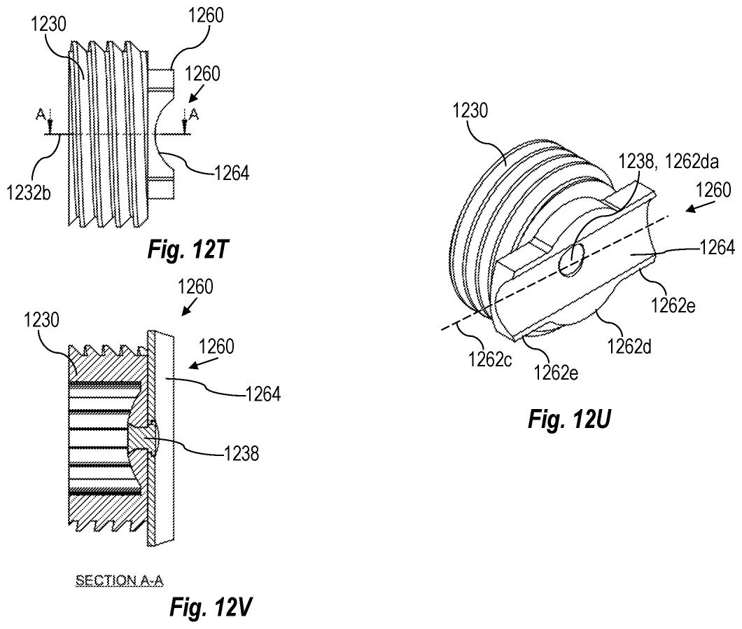

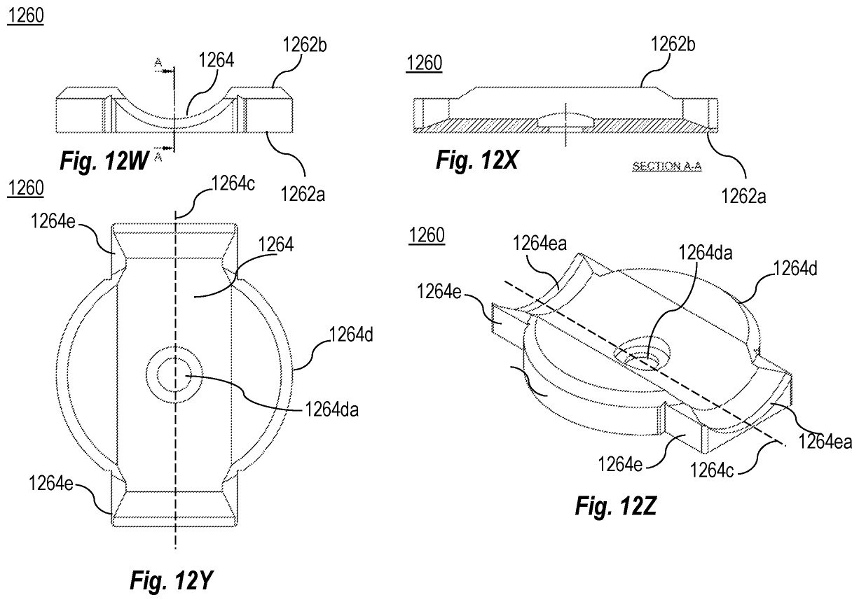

Reference is now made to FIGS. 12A, 12B, 12C and 12D, which are schematic illustrations of a device 1200 for connecting an orthopedic rod 90 to a pedicle screw 80, according to some embodiments of the invention.

FIG. 12A shows a perspective view of a disassembled device 1200 and of pedicle screw 80. FIGS. 12B and 12C show different, orthogonal side views of assembled device 1200 connected to pedicle screw 80 and orthopedic rod 90. FIG. 12D shows a cross-section view, along line A-A of FIG. 12C, of assembled device 1200 connected to orthopedic rod 90 and pedicle screw 80.

According to some embodiments of the invention, device 1200 for connecting an orthopedic rod 90 to a pedicle screw 80 includes a housing 1210, an adapter 1220, an internal bolt 1230, a spacer 1240 and restriction pins 1250. Housing 1210, adapter 1220, internal bolt 1230, spacer 1240 and restriction pins 1250 according to some embodiments of the present invention are made of the composite material.

Housing 1210 may have a substantially annular body. Housing 1210 may include two opposite concave indents 1214a, 1214b at its proximal end 1212a, which indents extend in a longitudinal direction of housing 1210. Concave indents 1214a, 1214b may be shaped and sized to receive orthopedic rod 90. Housing 1210 may include an internal thread 1216 on an internal surface of a proximal portion 1212b of housing 1210.

In some embodiments, housing 1210 includes one or more longitudinal housing grooves 1217 on the external lateral surface of housing 1210. For example, housing 1210 may include two opposing longitudinal housing grooves 1217 (e.g., one longitudinal housing groove 1217 as shown in FIG. 12A, and another longitudinal groove on the opposing side of housing 1210 which is not visible in FIG. 12A). Longitudinal housing grooves 1217 may be used to, for example, connect a tool to housing 1210 (e.g., such as tool 1600 described hereinbelow).

In some embodiments, housing 1210 includes one or more housing pin openings 1218. For example, housing 1210 may include two opposing housing pin openings 1218 (e.g., as shown in FIG. 12A). Housing pin openings 1218 may be made through the external lateral surface of housing 1210. Each of pin openings 1218 may be structured to receive a portion of one of restriction pins 1250. In some embodiments, each of housing pin openings 1218 is positioned within one of housing grooves 1217.

Adapter 1220 may include two longitudinal halves 1221. Each of longitudinal halves 1221 may include a recess 1224. Longitudinal halves 1221 may be shaped and sized to embrace and lock at least a portion of a pedicle screw head within recesses 1224 thereof. Longitudinal halves 1221 may be structured to be tightly inserted into a distal portion 1212d of housing 1210. Each of longitudinal halves 1221 may include a concave indent 1226 at its proximal end shaped and may be sized to receive a portion of orthopedic rod 90.

In some embodiments, each of longitudinal halves 1221 includes one or more longitudinal adapter grooves 1229. When longitudinal halves 1221 are tightly inserted into distal portion 1212d of housing 1210, restriction pins 1250 may be inserted into longitudinal adapter grooves 1229 through housing pin openings 1218. Once inserted, restriction pins 1250 may be connected to housing 1210 within housing pin openings 1218. For example, restriction pins 1250 may be welded (e.g., ultrasonically welded or welded by heat) or glued to side walls of housing pin openings 1218 or connected to housing 1210 by mechanical means. When connected within housing pin openings 1218, restriction pins 1250 may ensure proper positioning of adapter 1220 within distal portion 1212d of housing 1210 and/or prevent or limit axial motion and rotation of adapter 1220 with respect to housing 1210. Some embodiments of adapter 1220 are described below with respect to FIGS. 12H, 12I, 12J and 12K.

Internal bolt 1230 may include an external thread 1234 that is configured to mate with internal thread 1216 of housing 1210. Internal bolt 1230 may be structured to be screwed into proximal portion 1212b of housing 1210. Some embodiments of internal bolt 1230 are described below with respect to FIGS. 12L, 12M, 12N and 12O.

Spacer 1240 may be shaped and sized to be inserted into housing 1210 between adapter 1220 and internal bolt 1230. Spacer 1240 may be shaped and sized to be pressed by internal bolt 1230 against orthopedic rod 90 when internal bolt 1230 is being screwed into housing 1210. Various embodiments of spacer 1240 and spacer 1260 are described below with respect to FIGS. 12L-12S, FIGS. 12T-12V and FIGS. 12W-12Z.

In some embodiments, device 1200 has no spacer 1240. In embodiments in which device 1200 has no spacer 1240, orthopedic rod 90 is locked between adapter 1220 and internal bolt 1230 when internal bolt 1230 is tightly screwed into proximal portion 1212b of housing 1210.

A sequence of functions to be performed to connect orthopedic rod 90 to a pedicle screw 80 using device 1200 may include, at a first stage, embracing and locking pedicle screw head 82 between longitudinal halves 1221 of adapter 1220; at a second stage, tightly inserting longitudinal halves 1221 of adapter 1220 with pedicle screw head 82 locked therebetween into distal portion 1212d of housing 1210; and at a third stage, inserting restriction pins 1250 through housing pin openings 1218 and, for example, ultrasonically welding restriction pins 1250 within housing pin openings 1218. The sub-assembly of pedicle screw 80, housing 1210 and adapter 1220 may be inserted into a vertebra of a subject by a tool such as, for example, tool 1400 described hereinbelow. The sequence may include, at a fourth stage, inserting orthopedic rod 90 into concave idents 1214a, 1214b of housing 1210 and positioning orthopedic rod 90 into concave indents 1226 of longitudinal halves 1221 of adapter 1220; at a fifth stage, screwing internal bolt 1230 into proximal portion 1212b of housing 1210 so as to receive orthopedic rod 90 within a concave indent of spacer 1240; and at a sixth stage, tightening internal bolt 1230 within housing 1210.

Reference is now made to FIGS. 12E, 12F and 12G, which are schematic illustrations of a housing 1210 of device 1200 for connecting an orthopedic rod to a pedicle screw, according to some embodiments of the invention.

FIG. 12E shows a schematic perspective view of housing 1210. FIG. 12F shows a schematic partial section view of housing 1210. FIG. 12G shows a schematic section view of housing 1210.

Housing 1210 may be made of the composite material. Housing 1210 may have a substantially annular body and may have a proximal end 1212a, a proximal portion 1212b, a distal end 1212c and a distal portion 1212d.

Housing 1210 may include a first concave indent 1214a and a second concave indent 1214b at its proximal end 1212a. First concave indent 1214a and second concave indent 1214b may be in a longitudinal direction 1212e along housing 1210. First concave indent 1214a and second concave indent 1214b may be opposite to each other on housing 1210. First concave indent 1214a and second concave indent 1214b may be shaped and sized to receive an orthopedic rod.

In some embodiments, an internal surface 1212da of distal portion 1212d of housing 1210 tapers in longitudinal direction 1212e of housing 210. Internal surface 1212da of distal portion 1212d may be shaped and sized to tightly receive adapter 1220.

Housing 1210 may include an internal thread 1216. Internal thread 1216 may be on an internal surface of proximal portion 1212b of housing 1210.

In some embodiments, internal thread 1216 is coated with a coating material. The coating material may, for example, include PEEK or metal. The coating layer may have a thickness of, for example, 2.5-40 μm. The coating material may, for example, reduce a friction coefficient of the threads.

In some embodiments, internal thread 1216 is tilted at a predefined angle relative to a plane that is perpendicular to longitudinal direction/central longitudinal axis 1212e of housing 1210. Tilting of internal thread 1216 may, for example, create a force vector towards longitudinal direction/central longitudinal axis 1212e of housing 1210. This may, for example, prevent unintended opening of proximal end 1212a of housing 1210 when internal bolt 1230 is screwed therein. External thread 1234 of internal bolt 1230 may be tilted to mate with tilted internal thread 1216 of housing 1210.

In some embodiments, housing 1210 includes one or more longitudinal housing grooves 1217 on the external lateral surface of housing 1210. For example, housing 210 may include two opposing longitudinal housing grooves 1217. Longitudinal housing grooves 1217 may be used to, for example, connect housing 1210 to a tool such as tool 1600 described hereinbelow.

In some embodiments, housing 1210 includes one or more housing pin openings 1218 formed through the external lateral surface of housing 1210. For example, housing 1210 may include two opposing housing pin openings 1218 (e.g., as shown in FIG. 12E). Each of pin openings 1218 may be structured to receive one of restriction pins 1250. In some embodiments, each of housing pin openings 1218 is positioned within one of housing grooves 1217.

Restriction pins 1250 may ensure proper positioning of adapter 1220 within distal portion 1212d of housing 1210 and/or prevent or limit rotation axial motion and of adapter 1220 with respect to housing 1210 when restriction pins 1250 are connected (e.g., ultrasonically welded) to housing 1210.

In some embodiments, housing 1210 includes a chopped composite material.

In some embodiments, housing 1210 includes composite material fibers being arranged in at least one of: an axial direction of housing 1210, a radial direction of housing 1210, clockwise helical direction along housing 1210, counterclockwise helical direction along housing 1210, or any combination thereof. In some embodiments, composite material fibers are continuously arranged (e.g., side by side) along at least a portion the length of housing 1210. For example, composite material fibers may be applied in fragments separated by a distance of one or more fibers with respect to each other. In some embodiments, fibers of the composite material are evenly arranged in layers on at least a portion of the surface of the housing (e.g., top to bottom and through the circumference of the housing). In some embodiments, fibers of the composite material are arranged in uneven layering, for example, half height of only, 25% of the circumference or different thickness.

In some embodiments, housing 1210 includes two or more layers of composite materials. Fibers of the composite material may be arranged in different directions in each of the two or more layers.

For example, housing 1210 may include an internal composite material layer 1219a, an intermediate composite material layer 1219b and an external composite material layer 1219c (e.g., as shown in FIG. 12F). Fibers of the composite material may be arranged in different directions in internal composite material layer 1219a, in intermediate composite material layer 1219b and in external composite material layer 1219c (e.g., as shown in FIG. 12F). For example, in embodiments shown in FIG. 12F, in internal composite material layer 1219a fibers of the composite material may be arranged in a radial direction, in intermediate composite material layer 1219b fibers of the composite material may be helically arranged along the length of housing 1210 (the direction of helix may be, for example, clockwise, counterclockwise or a combination thereof), and in external composite material layer 1219b fibers of the composite material may be arranged at least partly in an axial direction (e.g., substantially along longitudinal direction 1212e of housing 1210).

In another example, in internal composite material layer 1219a and in external composite material layer 1219, fibers of the composite material may be helically arranged along the length of housing 1210 (the direction of helix may be, for example, clockwise, counterclockwise or a combination thereof).

It is noted that housing 1210 may include different number of layers, and the orientation of the composite material fibers in these layers may be different than shown and/or described in FIG. 12F. For example, embodiments described above with respect to FIG. 2F are also possible.

Reference is now made to FIGS. 12H, which is a schematic illustration of an adapter 1220 of device 1200 for connecting an orthopedic rod to a pedicle screw, according to some embodiments of the invention.

Reference is also made to FIGS. 12I, 12J and 12K, which are schematic illustrations of a longitudinal half 1221 of adapter 1220 of device 1200 for connecting an orthopedic rod to a pedicle screw, according to some embodiments of the invention.

FIG. 12H shows a schematic perspective view of adapter 1220. FIG. 12I shows a schematic perspective view of a longitudinal half 1221 of adapter 1220. FIGS. 12J and 12K show different schematic side views of longitudinal half 1221 of adapter 1220.

Adapter 1220 may include two longitudinal halves 1221. Each of longitudinal halves 1221 may be made of the composite material. Each of longitudinal halves 1221 may have a proximal end 1222a, a distal end 1222c, a flat longitudinal surface 1222e and a curved longitudinal surface 1222f.

Longitudinal halves 1221 may be shaped and sized to be tightly inserted into an interior 1212f of housing 1210 when longitudinal halves 1221 are positioned in proximity to each other and are aligned with respect to their flat longitudinal surfaces 1222e thereof. For example, longitudinal halves 1221 may be shaped and sized to be tightly inserted into distal portion 1212d of housing 1210 when longitudinal halves 1221 are positioned in proximity to each other and to be aligned with respect to their flat longitudinal surfaces 1222e. In some embodiments, curved longitudinal surfaces 1222f of each of longitudinal halves 1221 taper in a longitudinal direction 1222g of the respective halve. The tapering of curved longitudinal surfaces 1222f of longitudinal halves 1221 may mate with the tapering of internal surface 1212da of distal portion 1212d of housing 1210.

Each of longitudinal halves 1221 may include a recess 1224 on flat longitudinal surface 1222e thereof. Recess 1224 of each of longitudinal halves 1221 may extend from distal end 1222c towards proximal end 1222a in longitudinal direction 1222g along a portion of a length of the respective longitudinal halve.

Longitudinal recesses 1224 of longitudinal halves 1221 may be shaped and sized so as to receive and lock at least a portion of a head of a pedicle screw when longitudinal halves 1221 are positioned in a proximity to each other and aligned with respect to their flat longitudinal surfaces 1222e.

In some embodiments, proximal end 1222a of each of longitudinal halves 1221 includes a concave indent 1226 in direction 1222h of the respective halve. Concave indent 1226 of each of longitudinal halves 1221 may extend along the entire transverse dimension 1222h of proximal end 1222a of the respective longitudinal halve. Concave indent 1226 of each of longitudinal halves 1221 may be shaped and sized to receive a portion of the orthopedic rod.

In some embodiments, each of longitudinal halves 1221 of adapter 1220 includes one or more longitudinal adapter grooves 1229 on the external curved longitudinal surfaces 1222f of respective longitudinal halve. For example, each of longitudinal halves 1221 of adapter 1220 may include two opposing longitudinal adapter grooves 1229. In some embodiments, opposing longitudinal adapter grooves 1229 of each of longitudinal halves 1221 are adjacent to proximal end 1222a of respective longitudinal half. In some embodiments, opposing longitudinal adapter grooves 1229 of each of longitudinal halves 1221 are adjacent to flat longitudinal surface 1222e of respective longitudinal halve.

When longitudinal halves 1221 are tightly inserted into distal portion 1212d of housing 1210, restriction pins 1250 may be inserted into longitudinal adapter grooves 1229 of longitudinal halves 1221 of adapter 1220 through housing pin openings 1218 on the external lateral surface of housing 1210. Once inserted, restriction pins 1250 may be connected to housing 1210 (e.g., ultrasonically welded to side walls of housing openings 1218). Restriction pins 1250 may ensure proper positioning of adapter 1220 within distal portion 1212d of housing 1210 and/or prevent or limit rotation axial motion and of adapter 1220 with respect to housing 1210 when restriction pins 1250 are connected within housing pin openings 1218.

In the embodiments shown in FIGS. 12H, 12I, 12J and 12K, adapter 1220 includes two longitudinal halves 1221. It is noted that, in some embodiments, adapted 1220 may include more than two longitudinal members structured to embrace and lock the pedicle screw head when tightly inserted into distal portion 1212d of housing 1212. For example, adapter 1220 may include four longitudinal members structured to embrace and lock the pedicle screw head when tightly inserted into distal portion 1212d of housing 1212.

Reference is now made to FIGS. 12L, 12M, 12N and 12O, which are schematic illustrations of an internal bolt 1230 and a spacer 1240 of device 1200 for connecting an orthopedic rod to a pedicle screw, according to some embodiments of the invention. FIGS. 12L, 12M and 12N show different schematic perspective views of internal bolt 1230 and spacer 1240. FIG. 120 shows a schematic sectional view of internal bolt 1230 and spacer 1240 along line A-A of FIG. 12M.

Reference is also made to FIGS. 12P, 12Q, 12R and 12S, which are schematic illustrations of spacer 1240 of device 1200 for connecting an orthopedic rod to a pedicle screw, according to some embodiments of the invention. FIGS. 12P, 12Q, 12R and 12S show different schematic views of spacer 1240.

Internal bolt 1230 may be made of the composite material. Internal bolt 1230 may have a substantially annular body and may have a proximal end 1232a and a distal end 1232b. Internal bolt 1230 may include an external thread 1234 on an external lateral surface of internal bolt 1230. Thread 1234 may be configured to mate with internal thread 1216 of housing 1210. A profile of internal thread 1216 of housing 1210 may be shaped so as to not cause (or substantially not cause) outward radial forces during screwing of internal bolt 1230. A profile of internal thread 1216 of housing 1210 may be shaped to cause internal radial forces towards axis 1232c during screwing of internal bolt 1230. In some embodiments, thread 1234 is coated with a coating material. The coating material may, for example, include PEEK or metal. The coating layer may have a thickness of, for example, 2.5-40 μm. The coating material may, for example, reduce a friction coefficient of thread 1234.

Internal bolt 1230 may include a tool connector 1236 configured to connect a screwing tool to internal bolt 1230. In some embodiments, tool connector 1236 is at proximal end 1232a of internal bolt 1230. Connector 1236, for example, have a shape of multi longitudinal slots or torx or a polynomic shape like hex.

In some embodiments, internal bolt 1230 includes composite material fibers being arranged in at least one of: an axial direction of the internal bolt, a radial direction of the internal bolt, winded at an angle with respect to the central longitudinal axis 1232c of internal bolt 1230 in clockwise direction (e.g., clockwise helix), winded at an angle with respect to the central longitudinal axis 1232c of internal bolt 1230 in counterclockwise direction (e.g., counterclockwise helix), or any combination thereof. In some embodiments, internal bolt 1230 includes two or more layers of the composite material. Fibers of the composite material may have different orientations in the two or more layers, for example like described hereinabove with respect to composite material layers of the housing.

In some embodiments, lubricant is applied on external thread 1234 of internal bolt 1230. Application of lubricant may, for example, reduce friction between the internal bolt 1230 and the housing 1210. The lubricant may, for example, include blood or sterile oil.

Spacer 1240 may be made of the composite material. Spacer 1240 may have an upper flat surface 1242a. In some embodiments, spacer 1240 may include at its bottom surface 1242b two longitudinal concave indents 1243a and 1243b. Longitudinal concave indents 1243a and 1243b may be perpendicular to each other. Longitudinal concave indents 1243a and 1243b may guide spacer 1240 when being positioned on the orthopedic rod.

In some embodiments, spacer 1240 is rotatably connected to internal bolt 1230. In some embodiments, spacer 1240 is connected at its center point 1242da to distal end 1232b of internal bolt 1230 and is rotatable with respect to internal bolt 1230 about a central longitudinal axis 1232c of internal bolt 1230. For example, spacer 1240 may be rotatably connected to internal bolt 1230 using a hinge 1238. Hinge 1238 may be made of, for example, PEEK.

Reference is now made to FIGS. 12T, 12U and 12V, which are schematic illustrations of internal bolt 1230 and a spacer 1260 of device 1200 for connecting an orthopedic rod to a pedicle screw, according to some embodiments of the invention. FIGS. 12T and 12U and 12B show different schematic perspective views of internal bolt 1230 and spacer 1260. FIG. 12V shows a schematic sectional view of internal bolt 1230 and spacer 1260 along line A-A of FIG. 12T.

Reference is also made to FIGS. 12W, 12X, 12Y and 12Z, which are schematic illustrations of spacer 1260 of device 1200 for connecting an orthopedic rod to a pedicle screw, according to some embodiments of the invention. FIGS. 12W, 12Y and 12Z show different schematic views of spacer 1260. FIG. 12X shows a schematic sectional view of spacer 1260 along line A-A of FIG. 12W.

Spacer 1260 may be substantially flat and may have an upper flat surface 1262a and a bottom flat surface 1262b. Spacer 1260 may include a concave indent 1264 on bottom flat surface 1262b thereof. Concave indent 1264 may extend along the entire longitudinal dimension 1264c of spacer 1260. Concave indent 1264 may be shaped and sized to receive a portion of the orthopedic rod.

In some embodiments, spacer 1260 includes a central circular portion 1262d. Central circular portion 1262d of spacer 1260 may be shaped and sized to be inserted into an interior of housing 1210.

In some embodiments, spacer 1260 includes two radial protrusions 1262e radially protruding from central circular portion 1262d. In some embodiments, radial protrusions 1262e are opposite to each other on spacer 1260. Radial protrusions 1262e of spacer 1260 may be shaped and sized to be inserted into first concave ident 1214a and second concave indent 1214b of housing 1210. In some embodiments, each of radial protrusions 1262e includes a sloped surface 1264ea at its respective end.

In some embodiments, upper flat surface 1262a of spacer 1260 is coated with a coating material. The coating material may, for example, include PEEK or metal (e.g., Titanium or Titanium alloy). The coating layer may have a thickness of, for example, 2.5-40 μm. The coating material may, for example, reduce a friction coefficient of between spacer 1260 and internal bolt 1230.

In some embodiments, spacer 1260 is rotatably connected to internal bolt 1230. In some embodiments, spacer 1260 is connected at its center point 1262da to distal end 1232b of internal bolt 1230 and is rotatable with respect to internal bolt 1230 about a central longitudinal axis 1232c of internal bolt 1230. For example, spacer 1260 may be rotatably connected to internal bolt 1230 using a hinge 1238. Hinge 1238 may be made of, for example, PEEK.

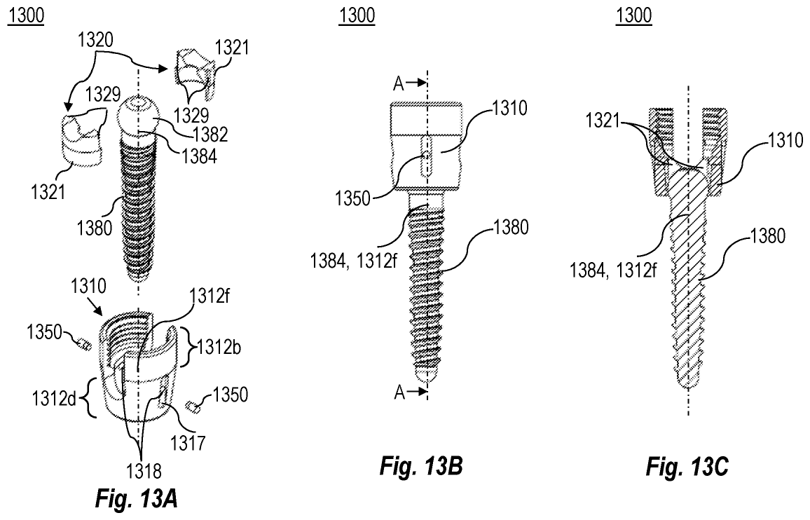

Reference is now made to FIGS. 13A, 13B and 13C, which are schematic illustrations of a pedicle screw sub-assembly 1300 including a housing 1310, an adapter 1320, restriction pins 1350 and a pedicle screw 1380, according to some embodiments of the invention.

FIG. 13A shows an exploded schematic perspective view of pedicle screw sub-assembly 1300. FIG. 13B shows a schematic side view of assembled pedicle screw sub-assembly 1300. FIG. 13C shows a schematic sectional view of assembled pedicle screw sub-assembly 1300 along line A-A of FIG. 13B.

According to some embodiments of the invention, pedicle screw sub-assembly 1300 includes a housing 1310, an adapter 1320 and a pedicle screw 1380. Housing 1310, adapter 1320 and pedicle screw 1380 may be made of the composite material.

Housing 1310 may be similar to housing 1210 of device 1200 described above with respect to FIGS. 12E, 12F and 12G. Housing 1310 may have a substantially annular body, a proximal portion 1312b, a distal portion 1312d, a central longitudinal axis 1312f, longitudinal housing grooves 1317 and housing pin openings 1318.

Adapter 1320 may be similar to adapter 1220 of device 1200 described above with respect to FIGS. 12H, 12I, 12J and 12K. Adapter 1320 may include two longitudinal halves 1321. Longitudinal halves 1321 may be positioned within distal portion 1312d of housing 1310 and may embrace and lock at least a portion of a head 1382 of pedicle screw 1380 such that a central longitudinal axis 1384 of pedicle screw 1380 coincides with a central longitudinal axis 1312f of housing 1310. Longitudinal halves 1321 of adapter 1320 are prepressed in a longitudinal direction of housing 1310 so as to maintain the coincidence of central longitudinal axis 1384 of pedicle screw 1380 with central longitudinal axis 1312f of housing 1310.

In some embodiments, each of longitudinal halves 1321 of adapter 1320 includes one or more longitudinal adapter grooves 1329 on the external lateral surface of respective longitudinal halve. For example, each of longitudinal halves 1321 of adapter 1320 may include two opposing longitudinal adapter grooves 1329. When longitudinal halves 1321 are tightly inserted into distal portion 1312d of housing 1310 and being prepressed, restriction pins 1350 may be inserted into longitudinal adapter grooves 1329 of longitudinal halves 1321 of adapter 1320 through housing pin openings 1318 on the external lateral surface of housing 1310. When connected to housing 1210 (e.g., ultrasonically welded to side walls of housing pin openings 1318), restriction pins 1350 may ensure proper positioning of adapter 1320 within distal portion 1312d of housing 1310 and/or may prevent or limit rotation of adapter 1320 with respect to housing 1310.

Pedicle screw sub-assembly 1300 may be assembled at, for example, manufacturer site. Pedicle screw sub-assembly 1300 may be implanted into a vertebra of a subject using a surgical tool. In various embodiments, pedicle screw sub-assembly 1300 may include an internal bolt screwable into housing 1310 and/or an internal bolt with a spacer. For example, the internal bolt and/or spacer may be similar to internal bolt 1230 and spacers 1240 described above with respect to FIGS. 12L-12O, 2P-12S, 12T-12V and 12W-12Z.

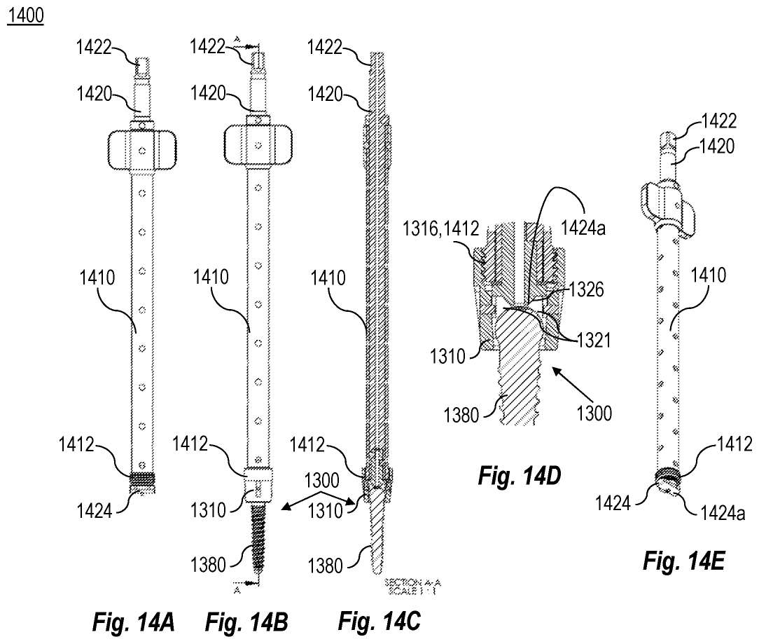

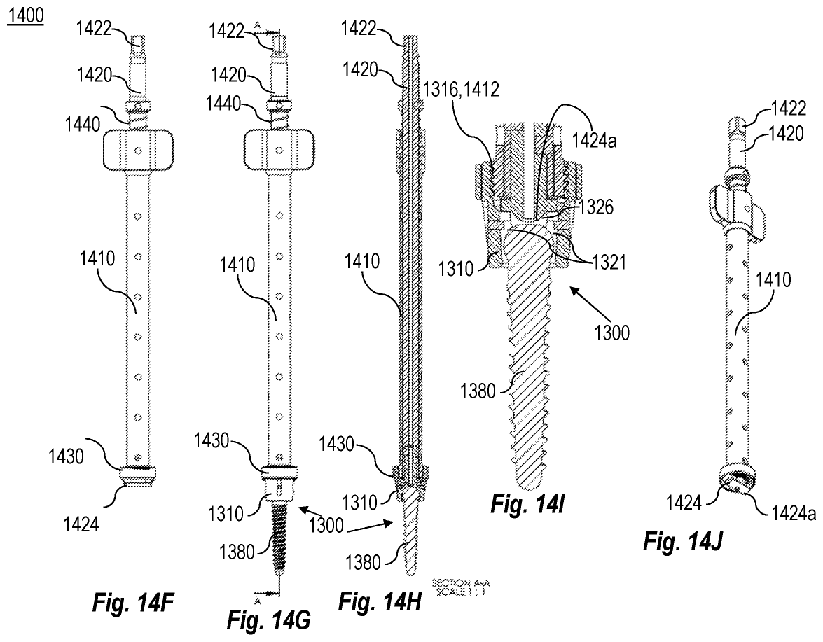

Reference is now made to FIGS. 14A, 14B, 14C, 14D and 14E, which are schematic illustrations of a tool 1400 for screwing pedicle screw sub-assembly 1300 into a vertebra of a subject, according to some embodiments of the invention.

Reference is also made to FIGS. 14F, 14G, 14H, 14I and 14J, which are schematic illustrations of tool 1400 including a ring member 1430 and a spring 1440, according to some embodiments of the invention.

FIGS. 14A, 14B, 14F, 14G show schematic side views of tool 1400. FIGS. 14C, 14D, 14H and 14I show schematic section A-A view of tool 1400. FIGS. 14E and 14J show perspective view of tool 1400.

According to some embodiments of the invention, tool 1400 includes a tube 1410 and a rod 1420. Rod 1420 may be positioned within tube 1410. In some embodiments, rod 1420 is cannulated, for example for k-wire use.

Tube 1410 may include an external thread 1412 at its distal end. External thread 1412 of tube 1410 may be configured to mate with the internal thread 1316 of housing 1310 of pedicle screw sub-assembly 1300 such that pedicle screw sub-assembly 1300 may be screwed to the distal end of tube 1410.

Rod 1420 may include a handle connector 1422 at its proximal end to connect a handle to rod 1420. In some embodiments, rod 1420 includes a holder 1424 at its distal end. Holder 1424 may be configured to hold pedicle screw sub-assembly 1300 while it is being screwed into the vertebra of the subject. In some embodiments, holder 1424 includes a longitudinal concave surface 1424a configured to mate with concave indents 1326 of longitudinal halves 1321 of adapter 1320 of pedicle screw sub-assembly 1300. When tube 1410 is screwed to pedicle screw sub-assembly housing 1310, tube 1410 applies force on holder 1424, which applies force on longitudinal halves 1321 of adapter 1320 to increase friction between adapted 1320 and the pedicle screw head and to transfer torque to the pedicle screw.

In some embodiments, tool 1400 includes a ring member 1430 connected to tube 1410 and surrounding the distal end of tube 1410 such that a gap is formed between an inner lateral surface of ring member 1430 and an external lateral surface of tube 1410. Ring member 1430 may lead insertion of pedicle screw sub-assembly 1300 to tool 1400. For example, ring member 1430 may ensure proper positioning of pedicle screw sub-assembly 1300 with respect to tool 1400 when pedicle screw sub-assembly 1300 being screwed to screwdriver tool 1400. Ring member 1430 may, for example, prevent radial expansion of housing 1310.

In some embodiments, tool 1410 includes a spring 1440. Spring 1440 may be loaded between rod 1420 and tube 1410. When pedicle screw sub-assembly 1300 is inserted into tool 1400, spring 1440 may push tube 1410/ring member 1430 with respect to rod 1420 to, for example, minimize an effort associated with the insertion action.

Pedicle screw sub-assembly 1300 may be connected to tool 1400 and may be further screwed into the vertebra of the subject using tool 1400.

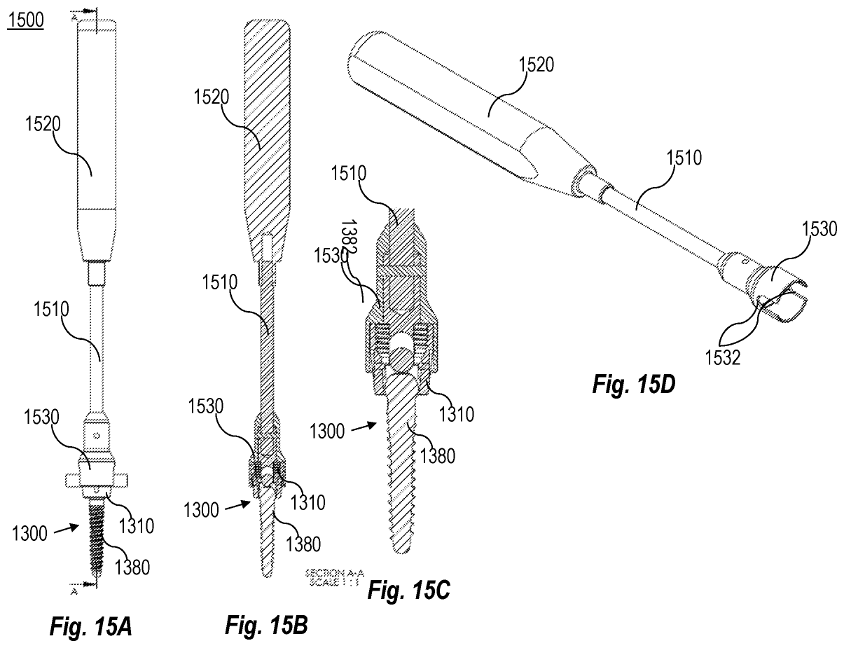

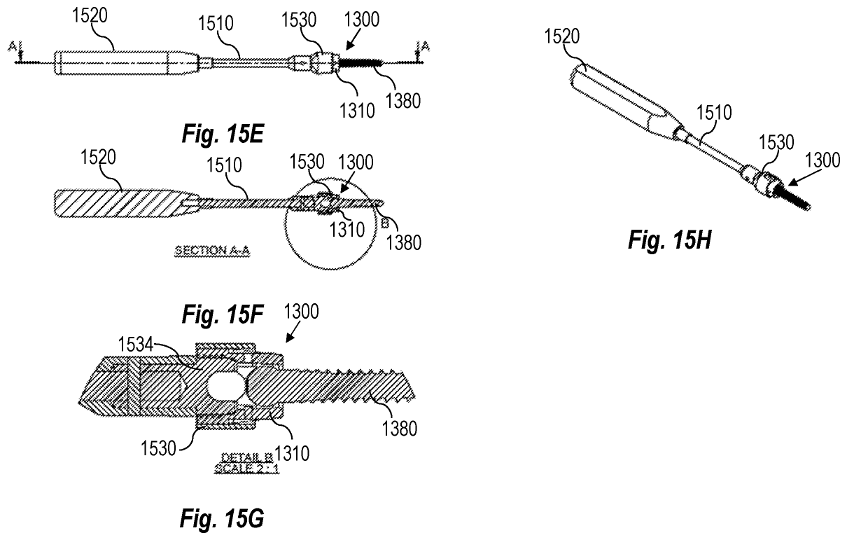

Reference is now made to FIGS. 15A, 15B,15C and 15D, which are schematic illustrations of a tool 1500 for loosening of a coupling of housing 1310/adapter 1320 with pedicle screw 1380 of pedicle screw sub-assembly 1300, according to some embodiments of the invention. FIG. 15A shows a schematic side view of tool 1500. FIGS. 15B and 15C show a schematic section A-A view of tool 1500. FIG. 15D shows a schematic perspective view of tool 1500.

Pedicle screw sub-assembly 1300 may include housing 1310, adapter 1320 having two longitudinal halves 1321 embracing and locking a portion of pedicle screw head 1382, wherein longitudinal halves 1321 of adapter 1320 are prepressed in a longitudinal direction of housing 1310 so as to maintain the coincidence of the central longitudinal axis of pedicle screw 1380 with the central longitudinal axis of housing 1310 (e.g., as described hereinabove). After pedicle screw sub-assembly 1300 is screwed into the vertebra of the subject, there may be a need to loosen the coupling of housing 1310/adapter 1320 with pedicle screw 1380 to, for example, allow adjusting a position and/or angle and/or orientation of housing 1310 with respect to pedicle screw 1380. The loosening may be performed using tool 1500.

Tool 1500 may include a rod 1510 having a handle 1520 at its proximal end and a gripper 1530 at its distal end. Gripper 1530 may grip or receive (e.g., tightly grip or receive) at least a portion of housing 1310 of pedicle screw sub-assembly 1300. In some embodiments, housing gripper 1530 includes two opposing concave indents 1532 at its distal end. Concave indents 1532 may be configured to receive portions of an orthopedic rod being accommodated within housing 1310.

Once gripper 1530 is connected to housing 1310, handle 1520 of tool 1500 may be moved to loosen the coupling of housing 1310/adapter 1320 with pedicle screw 1380. Upon loosening of the coupling of housing 1310/adapter 1320 with pedicle screw 1380, tool 1500 may be used to rotate housing 1310 with respect to pedicle screw 1380 in different directions so as to position housing 1310 at a desired position and/or orientation with respect to pedicle screw 1380. For example, tool 1500 may be used to change an angle between the longitudinal axes of housing 1310 and pedicle screw 1380.

Reference is now made to FIGS. 15E, 15F, 15G and 15H, which are schematic illustrations of the coupling loosening tool 1500 with gripper 1530 including an internal member 1534, according to some embodiments of the invention.

In some embodiments, housing gripper 1530 includes an internal member 1534. Internal member 1534 may be configured to be inserted into the proximal portion of pedicle screw sub-assembly housing 1310 so as to further support housing 1310 during handling of housing 1310 using tool 1500.

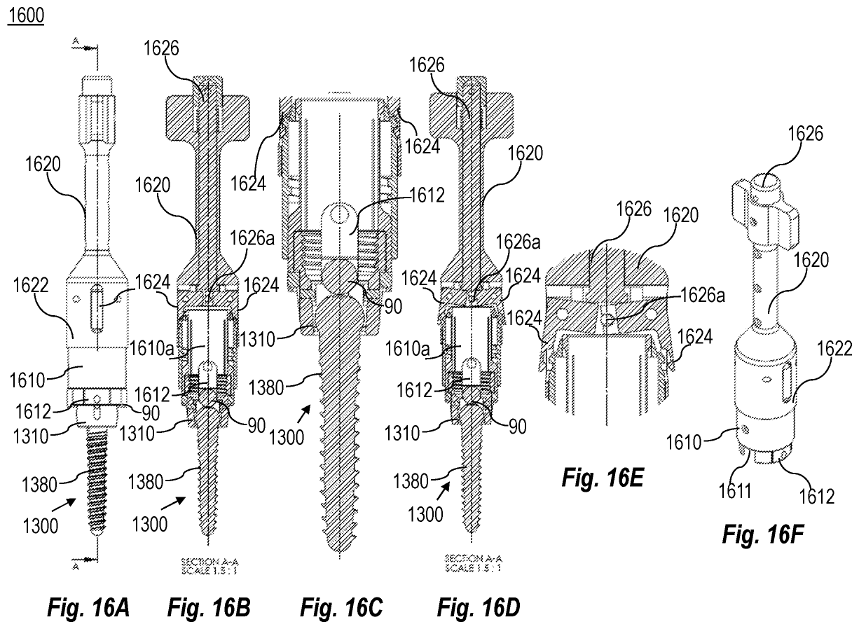

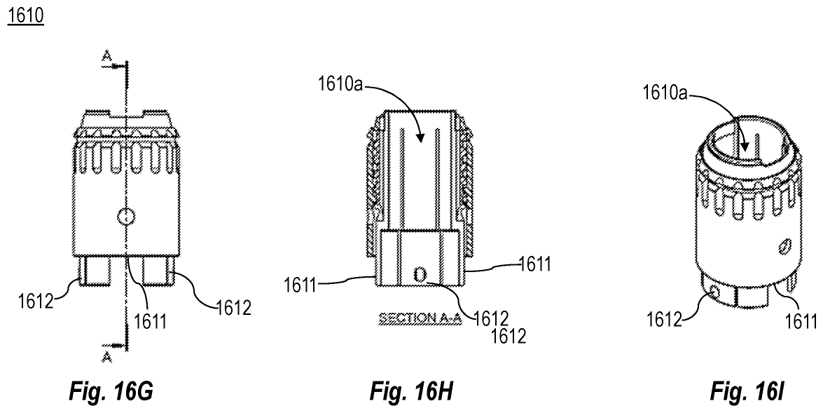

Reference is now made to FIGS. 16A, 16B, 16C, 16D, 16E and 16F which are schematic illustrations of a tool 1600 for positioning and holding an orthopedic rod 90 into a housing 1310 of pedicle screw sub-assembly 1300, according to some embodiments of the invention.

FIG. 16A shows a schematic side view of tool 1600. FIGS. 16B, 16C, 16D, 16E and 16F show a schematic sectional view of tool 1600 along line A-A of FIG. 16A. FIG. 16F shows a schematic perspective view of tool 1600.

Reference is also made to FIGS. 16G, 16H and 161, which are schematic illustrations of a gripper 1610 of the orthopedic rod positioning and holding tool 1600, according to some embodiments of the invention.

FIG. 16G shows a schematic side view of gripper 1610. FIG. 16H shows a schematic cross-section view, along line A-A of FIG. 16G, of gripper 1610. FIG. 16I shows a schematic perspective view of gripper 1610.

According to some embodiments of the invention, tool 1600 includes a gripper 1610 and a handle 1620.

Gripper 1610 may grip or receive pedicle screw sub-assembly housing 1310 and temporary hold orthopedic rod 90 within housing 1310. Gripper 1610 may include two opposing indents 1611 at its distal end, which indents 1611 are structured to receive and hold the orthopedic rod 90. In some embodiments, gripper 1610 includes two opposing gripper clips 1612 at its distal end. Gripper clips 1612 may be retractable. Gripper clips 1612 may releasably lock housing 1310 of pedicle screw sub-assembly 1300. When pedicle screw sub-assembly housing 1310 is inserted into gripper 1610, gripper clips 1612 may be received within, for example, longitudinal housing grooves 1317 of housing 1310 to lock housing 1310 and hold the orthopedic rod 90 within housing 1310. Gripper clips 1612 may be retracted to release housing 1310.

In some embodiments, handle 1620 is detachably connectable to gripper 1610. Handle 1620 may include a gripper housing 1622 that is structured to receive a portion of gripper 1612. Handle 1620 may include two opposing handle clips 1624. Handle clips 1624 may be retractable. Handle clips 1624 may releasably lock gripper 1610 within gripper housing 1612. Handle clips 1622 may be retractable. When gripper 1620 is received within gripper housing 1622 of handle 1620, handle clips 1624 may lock gripper 1610. For example, gripper clips 1624 and gripper 1612 may include mating dents and protrusions configured to lock gripper 1610 within gripper housing 1622. Handle clips 1624 may be retracted to release gripper 1610 from gripper housing 1622.

In some embodiments, handle 1620 includes a spring-loaded rod 1626 positioned within an interior of handle 1620. Spring-loaded rod 1626 may be coupled to handle clips 1624 at pivot 1626a at its distal end. Pressing of spring-loaded rod 1626 into the interior of handle 1620 may cause handle clips 1624 to retract. Releasing the pressure from spring-loaded rod 1626 may cause handle clips 1624 to draw back to their initial position.

In operation, gripper 1610 may be positioned onto pedicle screw sub-assembly housing 1310 using handle 1620 and may grip housing 1310 and temporary hold the orthopedic rod within housing 1310. Handle 1620 may be further detached from gripper 1610, and the internal bolt may be inserted through a hollow interior 1610a of gripper 1610. The internal bolt may be further screwed into pedicle screw sub-assembly housing 1310 to lock the orthopedic rod 90 within housing 1310 (e.g., as described hereinabove). Gripper 1610 may be further detached from pedicle screw sub-assembly housing 1310.

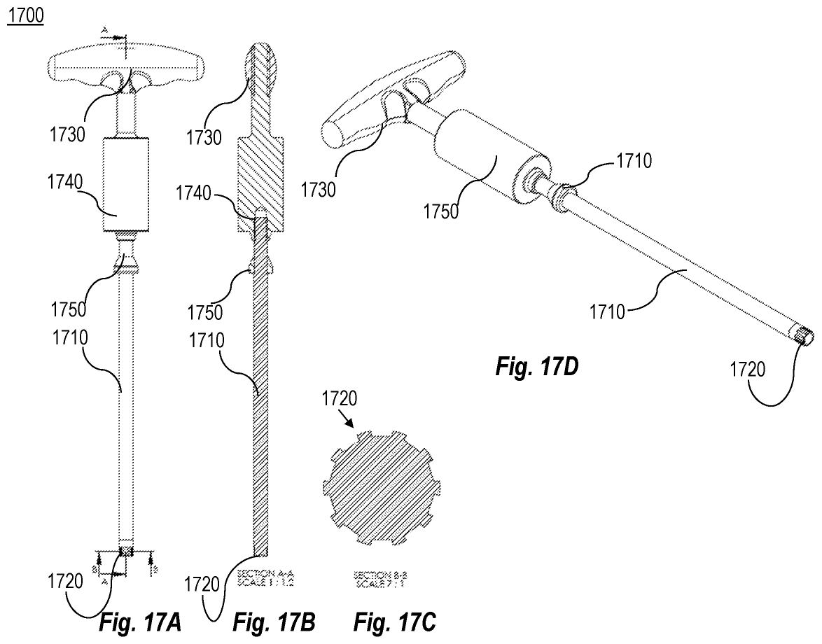

Reference is now made to FIGS. 17A, 17B, 17C and 17D, which are schematic illustrations of a tool 1700 for screwing an internal bolt into a housing of a pedicle screw assembly, according to some embodiments of the invention.

FIG. 17A shows a schematic side view of tool 1700. FIG. 17B shows a schematic section A-A view of tool 1700. FIG. 17C shows a schematic sectional view of bolt connector 1720 along line B-B of FIG. 17A. FIG. 17D shows a schematic perspective view of tool 1700.

Tool 1700 may include a rod 1710, an internal bolt connector 1720 at a distal end of rod 1710 and a handle 1730 at a proximal end of rod 1710. Internal bolt connector 1720 may be connected to the internal bolt. Tool 1700 may be used to screw the internal bolt into the housing of the pedicle screw sub-assembly (e.g., as described hereinabove). In some embodiments, tool 1700 includes a torque limiter 1740. In some embodiments, tool 1700 includes a stopper 1750. When tool 1700 is used with, for example, a counter torque tool (e.g., as described hereinbelow), stopper 1750 may serve as a position reference of tool 1700 with respect to the counter torque tool and/or as a pulling surface to release tool 1700 from the internal bolt.

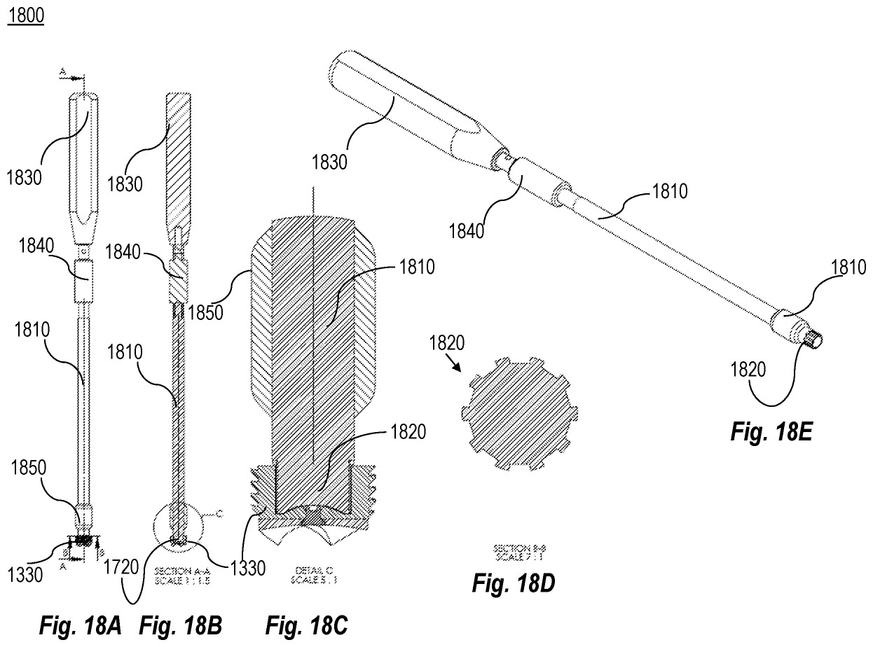

Reference is now made to FIGS. 18A, 18B, 18C, 18D and 18E, which are schematic illustrations of a tool 1800 for screwing an internal bolt 1330 into a housing of a pedicle screw assembly, according to some embodiments of the invention.

FIG. 18A shows a schematic side view of tool 1800. FIGS. 18B and 18C show schematic sectional views of tool 1800 along line A-A of FIG. 18A. FIG. 18D shows a schematic sectional view of bolt connector 1820 along line B-B of FIG. 18A. FIG. 18E shows a schematic perspective view of tool 1800.

Tool 1800 may include a rod 1810, an internal bolt connector 1820 at a distal end of rod 1810 and a handle 1830 at a proximal end of rod 1810. Internal bolt connector 1820 may be connected to the internal bolt. In some embodiments, internal bolt connector 1820 includes self-returning screwing head. Tool 1800 may be used to screw the internal bolt into the housing of the pedicle screw sub-assembly (e.g., as described hereinabove). In some embodiments, tool 1800 includes a torque limiter 1840. In some embodiments, tool 1800 includes an insertion lead member 1850 to lead the positioning of tool 1800, for example, when tool 1800 being used with tool 1600 described above.

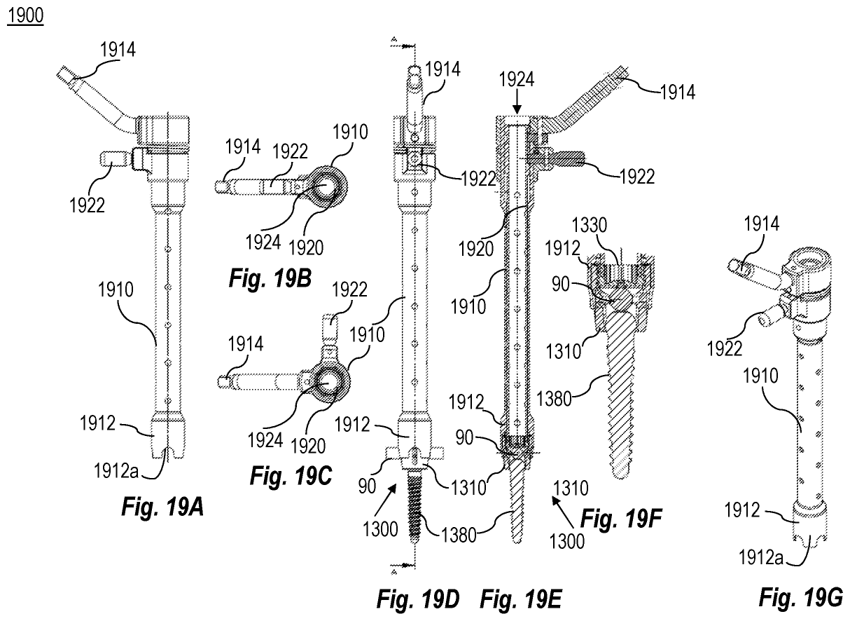

Reference is now made to FIGS. 19A, 19B, 19C, 19D, 19E, 19F and 19G, which are schematic illustrations of a tool 1900 for applying a counter torque, according to some embodiments of the invention.

FIGS. 19A and 19D show schematic side views of tool 1900. FIGS. 19B and 19C show schematic top views of tool 1900 with inner tube handle 1922 being in different positions. FIGS. 19E and 19F show schematic section views of tool 1900. FIG. 19G shows a schematic perspective view of tool 1900.

Tool 1900 may include an outer tube 1910 having a gripper 1912 at its distal end and a tool handle connector 1914 at its proximal end. Gripper 1912 may receive or grip housing 1310 of pedicle screw sub-assembly 1300. Gripper 1912 may include two or more pairs of opposing concave indents 1912a at its distal end to receive or grip orthopedic rod 90 therein. Tool handle connector 1914 may connect a tool handle to tool 1900.

Tool 1900 may include an inner tube 1920. Inner tube 1920 may be positioned within outer tube 1910. Inner tube 1920 may move in a longitudinal direction with respect to outer tube 1910. Inner tube 1920 may include an inner tube handle 1922. Inner tube handle 1922 may be moved within a spiral channel on the lateral surface of outer tube 1910. When moved in the spiral channel, inner tube handle 1922 causes inner tube 1920 to move in the longitudinal direction with respect to outer tube 1910. Longitudinal motion of inner tube 1920 with respect to outer tube 1910 may detach or release gripper 1912 from pedicle screw sub-assembly housing 1310.

In operation, gripper 1912 may receive or grip pedicle screw sub-assembly housing 1310 and orthopedic rod 90. Screwing tool such as, for example, tool 1700 described hereinabove may be inserted through a hollow interior 1924 of inner tube 1920 and connected to internal bolt 1330. A user may hold the tool handle connected to outer tube 1910 with one hand and operate the screwing tool with another hand to screw internal bolt 1330 to pedicle screw sub-assembly housing 1310. In this manner, tool 1900 may provide a counter torque to the user against orthopedic rod 90. Once the internal bolt 1330 is screwed into pedicle screw sub-assembly housing 1310, inner tube handle 1922 may be rotated to move inner tube 1920 in the longitudinal direction with respect to outer tube 1910 to detach or release gripper 1912 from pedicle screw sub-assembly housing 1310.

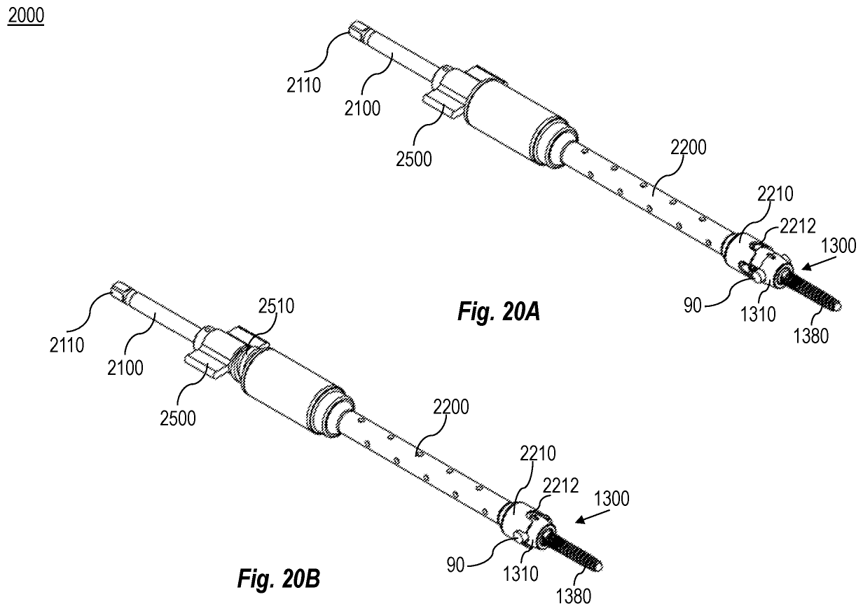

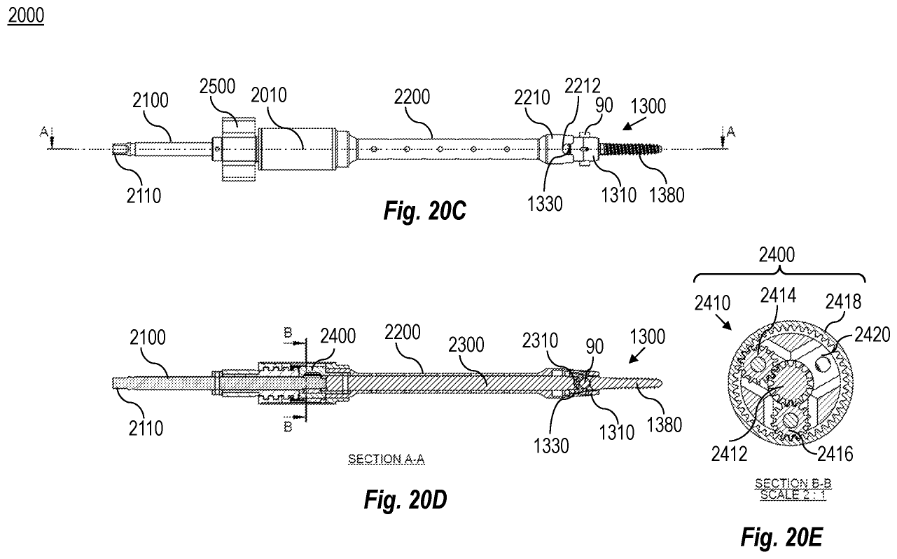

Reference is now made to FIGS. 20A, 20B, 20C, 20D and 20E, which are schematic illustrations of a tool 2000 for screwing an internal bolt into housing 310 of pedicle screw sub-assembly 1300, according to some embodiments of the invention.

According to some embodiments of the invention, tool 2000 includes an input rod 2100, a tube 2200, an output rod 2300 and a transmission 2400. Tool 2000 may have a central longitudinal axis 2010. Input rod 2100, tube 2200 and output rod 2300 may be aligned along central longitudinal axis 2010.

Input rod 2100 may include a handle connector 2110 at its proximal end. Input rod 2100 may be coupled at its distal end to transmission 2400, and output rod 2300 may be coupled at its proximal end to transmission 2400.

Tube 2200 may include a gripper 2210 at its distal end. Gripper 2210 may receive or grip housing 1310 of pedicle screw sub-assembly 1300. Gripper 2210 may include two or more pairs of opposing concave indents 2212 at its distal end to receive or grip orthopedic rod 90 therein.

Output rod 2300 may be positioned within tube 2200. Output rod 2300 may rotate within tube 2200. Output rod 2300 may be coupled at its proximal end to transmission 2400. Output rod 2300 may include a connector 2310 at its distal end to connect output rod to internal bolt 1310.

Transmission 2400 may transmit torque being applied on input rod 2100 to output rod 2300. Transmission 2400 may increase the torque being applied on input rod 2100. Transmission 2400 may be any type of transmission. For example, transmission 2400 may be from a family of epicyclic gearing or any other type that may get one input and two outputs.

In some embodiments, transmission 2400 includes a planetary gear assembly 2410. In some embodiments, gears of planetary gear assembly 2410 may rotate about axes that are parallel (or substantially parallel) to central longitudinal axis 2010 of tool 2000.

Planetary gear assembly 2410 may include a sun gear 2412. Sun gear 2412 may be rigidly connected to input rod 2100 to rotate with input rod 2100.

Planetary gear assembly 2410 may include one or more satellite gears that are meshed with sun gear 2412. In embodiments shown in FIG. 20A-20E, planetary gear assembly 410 includes three satellite gears each meshed with sun gear 2412. For sake of clarity, only two satellite gears 2414, 2416 are shown in FIG. 20E.

Planetary gear assembly 2410 may include a ring gear 2418. Ring gear 2418 may be meshed with the one or more satellite gears, for example with satellite gears 2414, 2416. Ring gear 2418 may be rigidly connected to tube 2200.

Transmission 2400 may include a cage 2420 to support the satellite gears.

The number of teeth on each of the gears of planetary gear assembly 2410 may be predefined based on a desired torque transmission ratio of transmission 2400. For example, transmission 2400 may be configured increase input torque of 2 N·m being applied on input shaft 2100 to output torque of 7.5 N·m. In this example, sun gear 2412 may include sixteen (16) teeth, each of satellite gears 2414, 2416 may include fourteen (14) teeth, and ring gear 2418 may include forty-four (44) teeth. In this example, an overall tightening torque at an output of transmission 2400 (e.g., a torque converted to output rod 2300) is larger than a torque received from input rod 2100. This is balanced by a greater number of rotations being performed by internal rod 2100 than a number of rotations being performed by output rod 2300.

When screwing tool 2000 having transmission 2400 is used, a torque transmitted to pedicle screw 1380 has the same value and direction as the torque being applied on input rod 2100. For example, if a torque of 2 N·m is applied to input rod 2100 in a first screwing direction, the torque transmitted to pedicle screw 1380 is 7.5 N·m in the same direction. Accordingly, a counter torque of 2.5 N·m opposite to the first screwing direction should be applied in order to balance the torque being transmitted to pedicle screw 1380.

In some embodiments, tool 2000 includes a torque limiter.

In some embodiments, output rod 2300 is configured to move with respect to tube 2200 in the longitudinal direction. For example, screwing a handle 2500 on a thread 2510 may cause output rod 2300 to move with respect to tube 2200 in the longitudinal direction. Longitudinal motion 1910 of output rod 2300 may cause output rod 2300 to be detached or released from the pedicle screw sub-assembly internal bolt 1330.

In the above description, an embodiment is an example or implementation of the invention. The various appearances of “one embodiment”, “an embodiment”, “certain embodiments” or “some embodiments” do not necessarily all refer to the same embodiments. Although various features of the invention can be described in the context of a single embodiment, the features can also be provided separately or in any suitable combination. Conversely, although the invention can be described herein in the context of separate embodiments for clarity, the invention can also be implemented in a single embodiment. Certain embodiments of the invention can include features from different embodiments disclosed above, and certain embodiments can incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A composite material device for connecting an orthopedic rod to a pedicle screw, the device comprising:
a housing having a substantially annular body and comprising:
two opposite concave indents at a proximal end of the housing and in a longitudinal direction along the housing, the concave indents being configured to receive an orthopedic rod; and
an internal thread on an internal surface of a proximal portion of the housing;
an adapter configured to lock at least a portion of a pedicle screw head within a distal portion of the housing; and
an internal bolt comprising an external thread configured to mate with the internal thread of the housing, the internal bolt being configured to be screwed into the proximal portion of the housing so as to lock the orthopedic rod between the adapter and the internal bolt;
wherein the housing, the adapter and the internal bolt comprise composite material;
wherein the internal thread of the housing and the external thread of the internal bolt are tilted at a predefined angle relative to a plane that is perpendicular to a central longitudinal axis of the housing.

2. The device of claim 1, wherein the adapter comprises two longitudinal halves being configured to embrace and lock at least a portion of the pedicle screw head therebetween and to be tightly inserted into the distal portion of the housing when embracing the pedicle screw head.

3. The device of claim 2, wherein each of the longitudinal halves of the adapter comprises a flat longitudinal surface and a curved longitudinal surface.

4. (canceled)

5. The device of claim 4, wherein the longitudinal recesses of the longitudinal halves are configured to receive and lock at least a portion of the pedicle screw head when the longitudinal halves are in a proximity to each other and are aligned with respect to their flat longitudinal surfaces.

6. The device of claim 3,
wherein the curved longitudinal surface of each of the longitudinal halves tapers in the longitudinal direction of the respective longitudinal halve,
wherein an internal surface of the distal portion of the housing tapers in the longitudinal direction of the housing, and
wherein the tapering of the internal surface of the distal portion of the housing mates with the tapering of curved longitudinal surfaces of the longitudinal halves.

7. The device of claim 2, wherein each of the longitudinal halves of the adapter comprises a concave indent extending in a transverse dimension at a proximal end of the respective longitudinal halve and being configured to receive a portion of the orthopedic rod.

8. The device of claim 1, wherein the housing and the adapter are configured to at least limit rotational and axial motion of the adapter with respect to the housing when the adapter is received within the housing.

9. The device of claim 2, further comprising two restriction pins that are configured to at least limit rotational and axial motion of the adapter with respect to the housing when the adapter is received within the housing.

10. The device of claim 9,
wherein the housing comprises two opposing housing pin openings made through an external lateral surface of the housing,
wherein each of the longitudinal halves of the adapter comprises an adapter groove configured to receive a portion of one of the restriction pins through one of the housing pin openings, and
wherein each of the restriction pins is connectable within one of the housing openings.

11. The device of claim 1, further comprising a spacer configured to be inserted into the housing between the adapter and the internal bolt so that, when the internal bolt is screwed into the proximal portion of the housing, the internal bolt presses the spacer against the orthopedic rod between the adapter and the spacer.

12. The device of claim 11, wherein the spacer is substantially flat and has an upper flat surface and a bottom flat surface, wherein the spacer comprises a concave indent on the bottom flat surface thereof, and wherein the concave indent extends along a longitudinal dimension of the spacer and is configured to receive a portion of the orthopedic rod.

13. The device of claim 12, wherein the spacer comprises:
a central circular portion configured to be tightly inserted into an interior of the housing; and
two opposite radial protrusions radially protruding from opposing sides of the central circular portion, wherein the radial protrusions are configured to be tightly inserted into the first concave indent and the second concave ident of the housing.

14. (canceled)

15. (canceled)

16. (canceled)

17. The device of claim 11, wherein the spacer is connected at its center point to a distal end of the internal bolt.

18. (canceled)

19. (canceled)

20. (canceled)

21. (canceled)

22. The device of claim 1, wherein the housing comprises composite material fibers being arranged along at least a portion of the housing in at least one of: an axial direction of the housing, a radial direction of the housing, a clockwise helical direction along the housing, a counterclockwise helical direction along the housing, or any combination thereof.

23. The device of claim 22, wherein the housing comprises two or more layers of composite material fibers, wherein the composite material fibers in at least a portion of the two or more layers are arranged in different direction with respect to each other.

24. The device of claim 1, wherein the internal bolt comprises composite material fibers being arranged at along at least a portion of the internal bolt in at least one of: an axial direction of the internal bolt, a radial direction of the internal bolt, a clockwise helical direction along the internal bolt, a counterclockwise helical direction along the internal bolt, or any combination thereof.

25. The device of claim 24, wherein the internal bolt comprises two or more layers of composite material fibers, wherein the composite material fibers in at least a portion of the two or more layers are arranged in different direction with respect to each other.

26. A composite material pedicle screw sub-assembly, the pedicle screw sub-assembly comprising:
a pedicle screw having a head and a central longitudinal axis;
a housing having a substantially annular body, a proximal portion, a distal portion and a central longitudinal axis;
an adapter comprising two longitudinal halves positioned within the distal portion of the housing, wherein the longitudinal halves are configured to embrace and lock a portion of the pedicle screw head such that the central longitudinal axis of the pedicle screw coincides with the central longitudinal axis of the housing;
wherein the longitudinal halves of the adapter are prepressed in a longitudinal direction of the housing so as to maintain the coincidence of the central longitudinal axis of the pedicle screw with the central longitudinal axis of the housing.

27. The sub-assembly of claim 26, further comprising two restriction pins being configured to at least limit rotation of the longitudinal halves of the adapter with respect to the housing.

28. (canceled)

29. A composite material pedicle screw implant assembly comprising:
an orthopedic rod;
a pedicle screw; and
a device for connecting the orthopedic rod to the pedicle screw, the device comprising:
a housing having a substantially annular body and comprising:
two opposite concave indents at a proximal end of the housing and in a longitudinal direction along the housing, the concave indents being configured to receive an orthopedic rod; and
an internal thread on an internal surface of a proximal portion of the housing;
an adapter configured to lock at least a portion of a pedicle screw head within a distal portion of the housing; and
an internal bolt comprising an external thread configured to mate with the internal thread of the housing, the internal bolt being configured to be screwed into the proximal portion of the housing so as to lock the orthopedic rod between the adapter and the internal bolt;
wherein the internal thread of the housing and the external thread of the internal bolt are tilted at a predefined angle relative to a plane that is perpendicular to a central longitudinal axis of the housing;
wherein the orthopedic rod, the pedicle screw, the housing, the adapter and the internal bolt comprise composite material.

30-50. (canceled)
